(19) United States
(12) Patent Application Publication
(10) Pub. No.: US 2025/0241948 A1
(43) Pub. Date: Jul. 31, 2025

(54) Engineered Extracellular Vesicle Delivery Systems and Uses Thereof

(71) Applicants: Yong-Yu Jhan, College Station, TX (US); Corey J. Bishop, College Station, TX (US); Guillermo Palou, College Station, TX (US); Daniel Prasca-Chamorro, Cypress, TX (US)

(72) Inventors: Yong-Yu Jhan, College Station, TX (US); Corey J. Bishop, College Station, TX (US); Guillermo Palou, College Station, TX (US); Daniel Prasca-Chamorro, Cypress, TX (US)

(73) Assignee: The Texas A&M University System, College Station, TX (US)

(21) Appl. No.: 19/067,145

(22) Filed: Feb. 28, 2025

Related U.S. Application Data
(62) Division of application No. 17/501,759, filed on Oct. 14, 2021.
(60) Provisional application No. 63/091,392, filed on Oct. 14, 2020.

(51) Int. Cl. A61K 35/12; A61K 45/06; A61K 47/54; C12N 5/071

(52) U.S. Cl. CPC A61K 35/12; A61K 45/06; A61K 47/543; C12N 5/0602

(57) ABSTRACT

Provided herein is an engineered extracellular vesicle (eEV) and an extracellular vesicle delivery vehicle. The engineered extracellular vesicle is an isolated extracellular vesicle that has a membrane hybridized with lipids. The extracellular vesicle delivery vehicle is a lipid-hybridized extracellular vesicle with a nucleic acid loaded within the core, a multi-layered polyelectrolyte coating deposited around the lipid-hybridized extracellular vesicle and a therapeutic drug complexed to one of the layer of polyelectrolyte coatings. Also provided are methods for preparing an engineered extracellular vesicle, for preparing an extracellular vesicle delivery vehicle, for for treating a pathophysiological condition in a subject, and for co-delivering a nucleic acid and a therapeutic drug to a cell of interest.

### CROSS-REFERENCE TO RELATED APPLICATIONS

This divisional application claims benefit of priority under 35 U.S.C. § 120 of pending non-provisional application U.S. Ser. No. 17/501,759, filed Oct. 14, 2021, which claims benefit of priority under 35 U.S.C. § 119(e) of provisional application U.S. Ser. No. 63/091,392, filed Oct. 14, 2020, now abandoned, the entirety of all of which is hereby incorporated by reference.

### FEDERAL FUNDING LEGEND

This invention was made with government support under Grant Number DP2 EB026265 awarded by the National Institutes of Health. The government has certain rights in the invention.

### BACKGROUND OF THE INVENTION

### Field of the Invention

The present invention relates to the fields of extracellular vesicles (EVs) and therapeutic delivery vehicles and systems. More particularly, the present invention relates to synthetic lipid-doped extracellular vesicles engineered for delivering therapeutic agents for treatment of pathophysiological diseases.

### Description of the Related Art

Extracellular vesicles (EVs), such as exosomes and microvesicles, have been acknowledged for their potential use as a therapeutic for the past 17 years). EVs are naturally secreted by most cell types. Typically, exosomes, which are a sub-population of EVs, are in the range of 30-150 nm in diameter (4,5). Extracellular vesicles functionality can be tailored by manipulating their cell sources, or endogenous tailoring through genetic or metabolic engineering, or by exogenous tailoring methods involving packing payloads post-isolation of the vesicles (6,7). Increasingly more studies have indicated that extracellular vesicles present fewer safety issues compared to cell therapy strategies or other conventional drug delivery systems (8). Furthermore, the extracellular vesicles' functionality can be tailored by manipulating their cell sources, or endogenous tailoring through genetic or metabolic engineering, or by exogenous tailoring methods involving packing payloads post-isolation of the vesicles (6,7).

As such due to their natural role in facilitating intercellular communication, nano-sized extracellular vesicles possess a high payload capability to deliver signaling molecules, such as protein, mRNAs, and microRNAs. They also exhibit many biological advantages over other nanomaterials, such as low immunogenicity, high stability in circulation, excellent biological barrier permeability, and intrinsic capabilities for selective tissue-homing and endosomal escape or fusion (9). While most of the synthetic nanocarriers must be modified with specific ligands to enhance tumor targeting, extracellular vesicles derived from different host cells have extraordinary ability to selectively target cancer cells, which circumvents the need for surface modification for active targeting (10-13).

For example, combining chemotherapy with RNA interference (RNAi)-based therapy has attracted interest for overcoming multidrug resistance (MDR) in cancer (14) The concept of utilizing small-interfering RNA (siRNA) provides a strategy for selectively down-regulating the abnormal genes that confer resistance, such as anti-apoptotic genes (i.e., Bcl-2 and Survivin) and drug-efflux pumps (i.e., P-glycoprotein) (15). Several studies have demonstrated that an increased cell sensitivity to chemotherapeutic agents and higher tumor killing efficiency can be achieved by combinatorial treatment of doxorubicin (DOX) and siRNA (e.g., Bcl-2 (16), P-gp (17), PLK1 (18), confirming that a combinatorial drug-siRNA therapy can improve efficacy. Moreover, research has found that synchronous delivery of siRNA and chemotherapeutics from a single nanocarrier is more effective at treating cancer when compared to delivery via two separate nanocarriers (19). This finding suggests that the co-delivery of drugs to the same cell plays a critical role in the therapeutic efficacy (20,21).

However, it is difficult to encapsulate siRNA and chemotherapeutics in a single nanocarrier due to their different physicochemical properties. While siRNA exhibits high molecular weight (˜13-14 kDa) and highly negative-charged phosphate backbone, chemotherapeutic agents, such as DOX and paclitaxel, are relatively hydrophobic small molecules. A number of modifications to these particles have been proposed to improve the delivery efficiency, including conjugation of cell targeting ligands (22-24) combinations of different hydrophilic/hydrophobic segments (25) incorporation of pH-responsive moieties (26-28), and bio-reducible linkages (29) For most of these materials, the hydrophilic or hydrophobic chemotherapeutic agent is conjugated or loaded in the core via self-assembly, while the anionic siRNA is complexed with cationic macromolecules via electrostatic condensation, which leads to a core-shell structure delivery system. While significant improvements in therapeutic efficacy have been shown in some of these studies, there is still a need to develop combinatorial delivery systems with targeting capabilities. Moreover, one of the major hurdles is the lack of cost-effective methods to obtain sufficient quantities of EVs with consistent biochemical characteristics for clinical application (30,31). There is difficulty in scaling up EVs from a manufacturing standpoint; the yields for EV isolation and purification from in vitro cell culture are extremely low and often impractical.

Thus, there is a recognized need in the art for stable, effective, and more scalable extracellular vesicle-based formulations which can be loaded with exogenous cargo. Particularly, the prior art is deficient in an engineered polymer-coated extracellular vesicles-based platform that can selectively co-deliver both small molecule anti-cancer drugs and nucleic acids to cancer cells. The present invention fulfills this long standing need and desire in the art.

### SUMMARY OF THE INVENTION

The present invention is directed to an engineered extracellular vesicle (eEV). The eEV comprises an extracellular vesicle isolated from a biological cell with at least one lipid incorporated into the membrane thereof.

The present invention also is directed to a method for preparing an engineered extracellular vesicle. In the method the biological cell described herein is cultured in vitro in a culture medium and the extracellular vesicles are isolated from the biological cells. The isolated extracellular vesicles are extruded with the at least one lipid to form the engineered extracellular vesicle.

The present invention is directed further to an extracellular vesicle delivery vehicle. The extracellular vesicle delivery vehicle comprises at least one lipid hybridized with a membrane of the extracellular vesicle and a nucleic acid loaded within a core of the extracellular vesicle. A multi-layered polyelectrolyte coating is deposited around the extracellular vesicle and a therapeutic drug is complexed with the multi-layered polyelectrolyte coating.

The present invention is directed further still to a method for treating a pathophysiological condition in a subject in need of such treatment. In the method an amount of the extracellular vesicle delivery vehicle described herein is effective to at least decrease a population of cells associated with the pathophysiological condition is administered to the subject.

The present invention is directed further still to a method for co-delivering a nucleic acid and a therapeutic drug to a cell of interest. In the method the cell of interest is contacted with the extracellular vesicle delivery vehicle described herein.

The present invention is directed further still to a method for method for preparing an extracellular vesicle delivery vehicle. In the method biological cells are cultured in vitro in a culture medium and the extracellular vesicles are isolated from the biological cells. The isolated extracellular vesicles are extruded with at least one lipid to form a lipid-hybridized extracellular vesicle and a nucleic acid is loaded into a core of the lipid-hybridized extracellular vesicle. Sequentially, a first layer of a polycation is deposited to coat the lipid-hybridized extracellular vesicle, a second layer of a polyanion is deposited onto the first layer and a third layer of a cationic polymer ester is deposited onto the second layer. A therapeutic drug is complexed to the second layer to form the extracellular vesicle delivery vehicle.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others that will become clear, are attained and can be understood in detail, more particular descriptions of the invention briefly summarized above may be had by reference to certain embodiments thereof that are illustrated in the appended drawings. These drawings form a part of the specification. It is to be noted, however, that the appended drawings illustrate preferred embodiments of the invention and therefore are not to be considered limiting in their scope.

FIGS. 1A-1E characterize the extracellular vesicles (EVs) after ultracentrifugation. FIG. 1A is the relative size (diameter) distribution profiles for A549 (dashed line) and 3T3 cell (solid line)-derived EVs. FIG. 1B is a transmission electron microscope (TEM) image showing the spherical structure of EVs (3T3 EV; scale bar: 200 nm). FIG. 1C illustrates left y-axis reports the production yield of EVs secreted by A549 and 3T3 cells (left y-axis) and affiliated protein per EV (Micro BCA assay) (right y-axis). FIGS. 1D-1E show flow cytometric analysis of EVs stained with fluorescent labelled antibodies targeting CD63 in a scatter plot of A549 cell-derived EV (FIG. 1D, bottom right box) and the negative isotype control (FIG. 1D, bottom left) and in a histogram of CD63⁺ A549 EVs stained with anti-CD63-eFluor 660 and negative isotype control (* and *** represent p-values<0.05 and <0.001, respectively).

FIGS. 2A-2I shows the morphology, size, quantification of mass production and zeta potential measurement of engineered extracellular vesicles (eEVs). The size distribution profiles and morphology of vesicles for hydrated lipids POPC (FIGS. 2A-2B) and engineered EVs POPC-EV (FIGS. 2C-2D) were measured by NTA and TEM. Scale bar represents 100 nm. The mean diameters (FIG. 2E) of different lipid-doped eEVs were determined by NTA measurement. Fold increase in particle number (FIG. 2F) was quantified by normalizing the concentration of eEVs after extrusion processes to the concentration of original native EV concentration before extrusion processes. Zeta potential values measured in water and PBS are reported in (FIG. 2G). (*, **, ***, ****, represent p-values<0.05, <0.01, <0.001, and <0.0001, respectively). FIG. 2H is a particle number quantification of DOTAP, POPC, DPPC, and POPG lipids at 2.5 mM each by NTA measurement. FIG. 2I illustrates zeta potential measurement of POPC-EV with POPC:EV mixing ratio from 1:1 to 9:1 before (physical mixture in the absence of sonication-extrusion processes) and after sonication-extrusion processes.

FIGS. 3A-3F show the assessment of membrane incorporation of engineered extracellular vesicles (eEVs) via protein: lipid ratio quantification and FACS analysis. FIG. 3A shows the absorbance linearity of the lipids DOTAP, POPC, DPPC, POPG, and cholesterol. FIG. 3B shows the protein to lipid ratios of pure lipid PBS aqueous solutions of DOTAP, POPC, DPPC, and POPG. FIG. 3C shows the protein quantification of engineered extracellular vesicles POPC-EV (50 μg/ml EVs; 5 mM POPC) via Micro BCA protein assay (****p<0.0001). The protein: lipid ratios within the membrane of different lipid-doped eEVs were quantified in FIG. 3D. FIG. 3E are flow cytometry histograms A549 EVs stained with anti-CD63-eFluor 660 (solid line), eEV stained with anti-CD63-eFluor 660 (dotted line), and a stained isotype (dashed line) functioning as a negative control. FIG. 3F shows the normalized anti-CD63 geometric mean fluorescence intensities (GMFIs) and quantify the fusion ratio of synthetic lipids doped within EVs, thereby forming eEVs (0-1 represents 0%-100% native EV). (**** represents p-values<0.0001).

FIG. 4 shows the exogenous loading of siRNA via electroporation system. The left y-axis shows siRNA loading efficiency (black) and the right y-axis shows encapsulation efficiency (gray). (*** and **** represent p-values<0.001 and <0.0001, respectively).

FIGS. 5A-5F show the characterization of aggregation post-electroporation by spectroscopic assays and microscopy image analysis. FIG. 5A illustrates the cell viability of A549 cells incubated with various eEVs with brightfield images of the eEV and EV formulations (scale bar=100 μm). FIG. 5B shows the absorption spectra of siRNA (1 ng/μl) and EV/eEV (1010-1011 vesicles/ml) in Opti-MEM after electroporation. FIG. 5C shows the turbidity (OD: 230 nm) for EV, eEVs and siRNA conditions in the absence of siRNA (w/o siRNA, meaning only vesicles in the solution) or in the presence of siRNA (vesicle+siRNA) during electroporation. The absorbance values in y-axis have the background values (Opti-MEM media/pre-electroporated samples) subtracted. FIG. 5D is a log-log plot of turbidity (OD: 230 nm) versus vesicle concentration after electroporation. FIGS. 5E-5F are semi-log plots of aggregate counts per ml and aggregate size (diameter) versus vesicle concentration after electroporation. Data were determined by microscopy image analysis.

FIGS. 6A-6C show the effects of electroporation media on the formation of aggregates (FIG. 6A) and cell viability (FIG. 6B) including cell viability of eEVs on CCL-210 cells (FIG. 6C). Note: The cell viability assay was not conducted for eEVs in hypotonic electroporation buffer due to the excessively high level of aggregation.

FIGS. 7A-7G show the RNA interference knockdown of eEVs in lung tumor cells (A549). FIG. 7A is fluorescence images of delivered anti-GFP siRNA with eEVs to A549 cells at 3 days post-transfection. scale bar: 100 μm. FIGS. 7B-7B show the statistical analysis of overall knockdown efficiency of eEVs. FIG. 7B shows the effects of EV and eEVs in Opti-MEM+EDTA. FIG. 7C shows the effects of delivered dosages. POPG-EV in 100 μl Opti-MEM: 1-fold, 2-fold, and 4-fold are (10¹¹ particles/ml, 1 ng/μl siRNA), (2×10¹¹ particles/ml, 2 ng/μl siRNA) and (4×10¹¹ particles/ml, 4 ng/μl siRNA), respectively. FIG. 7D shows the effects of EV and in 50 mM trehalose. FIG. 7E shows the knockdown curves over time of lipid-siRNA complexes following electroporation. Same molecule concentration (2.5 μl of 2.5 mM) of lipids as the final amounts used for eEV-siRNA delivery was prepared. FIG. 7F shows the knockdown efficiency of POPG-EV with 1 to 4-fold amounts of dose in Opti-MEM (with EDTA addition following electroporation). FIG. 7G shows the knockdown efficiency of eEVs in Trehalose electroporation media.

FIGS. 8A-8C show the visualization and quantification of eEV uptake to lung adenocarcinoma (A549) and lung normal fibroblast (CCL-210). FIG. 8A is the confocal microscopy images of eEV (POPC-EV) incubated with A549 cells for 2 hours. Lower left is the DeepRed@dye for eEV membrane staining, upper left is the Hoechst 33342 nuclei staining, upper right is the GFP expression in cell cytoplasm of A549 cells, lower right is a merge. Scale bar: 30 μm; FIG. 8B shows the normalized geometric mean fluorescence intensities (GMFI) from flow cytometry analysis to compare the cellular uptake efficiency of eEVs in A549 and CCL-210. 10¹¹ particles of POPC-EV was used as representative of eEVs; same amounts of EVs (10¹¹ particles) were delivered to the cells to compare the uptake efficiency. FIG. 8C are flow cytometry histograms (FL4-H) of eEVs uptake by A549 and CCL-210 cells; Grey dashed peak: Untreated A549 cells; Grey long-dashed peak: Untreated CCL-210; Blue dotted peak: eEV uptake in CCL-210; Red solid peak: eEV uptake in A549.

FIGS. 9A-9M show the characterization of zeta potential, particle concentration and diameter for each sequential layer of LbL-eEVs. Preliminary data for deposition on native EV, zwitterionic POPC-doped eEV and anionic POPG-doped eEV via LbL assembly for zeta potential (FIG. 9A) and particle concentration changes with the addition of PLL in the concentration range of 0-100 μg/ml (FIG. 9B). Data after addition of the first layer: PLL (FIG. 9C-9E), second layer: PAA (FIGS. 9F-9H), and final layer: PBAE (FIG. 9I-9K) at varying polyelectrolyte concentrations is shown. FIG. 9L is a TEM showing vesicle morphology of immuno-gold labeled CD63 molecules. FIG. 9M shows CD63 characterization of LbL-eEV using on bead flow cytometry analysis.

FIGS. 10A-10D show the physicochemical characterization of the optimized LbL-eEVs after addition of each polyelectrolyte layer on the eEV core. FIGS. 10A-10B show the changes in zeta potential (FIG. 10A) and diameter (FIG. 10B) of LbL-eEVs. First layer: 200 μg/ml cationic poly (L-lysine) (PLL) in 150 mM NaAc. Second layer: 0.5 mg/ml anionic poly (acrylic acids) (PAA) in 150 mM NaAc. Final layer: 2.5 mg/ml cationic bio-reducible poly (β-amino ester) s (PBAE) (BR647) in 25 mM NaAc. ***p<0.001; FIGS. 10C-10D show the amount of drug loaded after LbL assembly. FIG. 10C shows the exogenous loading of siRNA into eEVs in various electroporation media. *p<0.05, **p<0.01, ***p<0.001; FIG. 10D shows the amount of DOX loaded within tri-layered polyelectrolyte shells of LbL-PBAE-eEVs using different drug concentrations. A logistic non-linear regression model was used for curve fitting. The loading amount of DOX per 10¹¹ vesicles was quantified by spectrophotometric method. A linear function of DOX standard concentrations versus fluorescence (Excitation: 485 nm/Emission: 590 nm) was used as the calibration curve.

FIGS. 11A-11C shows the visualization and quantification of LbL-eEV uptake in normal lung fibroblast (CCL-210) and lung adenocarcinoma cells (A549). FIG. 11A are confocal images of LbL-eEV staining with Deep Red dye in the cells. scale bar: 30 μm; FIG. 11B are flow cytometry histograms (FL4-H) of LbL-eEV uptake by A549 and CCL-210 cells. LbL-eEV in A549: Red, solid line; LbL-EV in CCL-210: black, dotted line; vesicles without dye in A549: blue, dashed line; vesicles without dye in CCL-210: gray, long-dashed line; FIG. 11C show normalized geometric mean fluorescence intensities (GMFI) indicating cellular uptake efficiency of LbL-eEVs in A549 and CCL-210 cells. ***p<0.001, ****p<0.0001.

FIGS. 12A-12F show the siRNA delivery and RNA interference knockdown with LbL-eEVs in CCL-210 and A549 cells. FIG. 12A are flow cytometry histograms (FL2-H) of Cy3-labelled siRNA uptake by A549 and CCL-210 cells; FIG. 12B shows normalized geometric mean fluorescence intensities (GMFI) of LbL-eEV mediated siRNA uptake efficiency in A549 and CCL-210 cells. Lipofectamine RNAiMAX with 50 ng siRNA was prepared as a positive control group. GMFI was obtained by normalizing to the untreated cells. ****p<0.0001; FIG. 12C shows siRNA-mediated GFP knockdown of LbL eEVs (PBAE-layered) quantified over time in A549 cells by fluorescence measurements on a Cytation 5 plate reader. OptiMEM+EDTA, 50 mM Trehalose and hypotonic electroporation buffer were used during electroporation for siRNA loading into the LbL-eEVs; FIG. 12D shows the overall knockdown efficiency (the area under the curves over time) of LbL-eEVs after different polyelectrolyte layers were added in A549 cells. OptiMEM+EDTA was used as the electroporation buffer. *p<0.05, *p<0.01, *p<0.001; FIG. 12E shows the overall knockdown efficiency in A549 cells at varying LbL eEV dose (1.875-60×10¹¹ vesicles/ml). FIG. 12F are confocal microscope images of cells treated by different formulations with Cy3-siRNA delivery with fluorescent images showed Cy3-siRNA in cell cytoplasm. Scale bar: 30 μm.

FIGS. 13A-13D show the DOX delivery and cancer killing effects in A549 and CCL-210 cells. FIG. 13A are confocal images of Free DOX and LbL-eEV mediated DOX delivery in A549 and CCL-210 cells, Scale bar: 30 μm; FIG. 13B show the normalized geometric mean fluorescence intensities (GMFI) of the DOX uptake efficiency in A549 and CCL-210 cells. PLGA nanoparticles (PLGA NP) were prepared for comparison. Equivalent amounts of DOX in LbL-PAA were prepared to compare with LbL-PBAE; FIGS. 13C-13D show the dose effects of free DOX, PLGA NP-delivered DOX, and LbL-eEV delivered DOX on (FIG. 13C) A549 and (FIG. 13D) CCL-210 cells after 3 days incubation. DOX concentration was calculated by: particle concentration (measured by NTA)×DOX loading/LbL-eEV particles.

FIGS. 14A-14D show the co-delivery of DOX and siRNA-GFP/siRNA-scramble in A549 cells with different formulations. FIG. 14A shows the fluorescence microscope images of cells treated for 3 days with different formulations. Scale bar: 50 μm FIGS. 14B-14D show the fluorescence signals from green fluorescence protein expression (GFP/FL1-H) and a dead cell stain (EthD-1/FL3-H) in A549 cells were measured by flow cytometry and presented as (FIG. 14B) four quadrant diagrams, (FIG. 14C) normalized geometric fluorescence mean (nGMFI) of FL3-H (**p<0.01), and (FIG. 14D) knockdown efficiency (FL1-H). No statistical difference of knockdown efficiency (FL1-A) was observed between all groups.

### DETAILED DESCRIPTION OF THE INVENTION

As used herein in the specification, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the terms “engineered extracellular vesicle”, “eEV”, “modified extracellular vesicle”, “lipid-hybridized extracellular vesicle”, and “hybridized extracellular vesicle”, are interchangeable and refer to an extracellular vesicle isolated from a biological cell and with a membrane hybridized to or modified with at least one lipid.

As used herein, the terms “LbL-eEV”, “layer-by-layer-eEV”, “layer-by-layer (LbL)-coated engineered extracellular vesicle” and “LbL-coated eEV” are interchangeable and refer to an engineered extracellular vesicle with multiple layers of cationic and anionic polyelectrolytes coated or layered on the eEV to form a shell therearound or to encapsulate the eEV. The polyelectrolyte layers are oppositely charged one from the other.

As used herein, the term “lipid” refers to a synthetic lipid or an exogenous or non-native lipid or a combination thereof.

As used herein, the term “nucleic acid” refers to any synthetic or naturally occurring DNA or RNA or fragments thereof.

As used herein, the term “therapeutic drug” refers to any pathophysiologically-treating agent or entity. Non-limiting examples are an anti-cancer drug, a protein such as an aptamer or antibody including a duobody or bispecific antibody, and a small molecule drug.

The following acronyms are used herein. 3T3 is a cell line of mouse embryonic fibroblast cells; A549 is a cell line of adenocarcinoma human alveolar basal epithelial cells; CCL-210 is a cell line of human normal lung fibroblast cells; DLS is dynamic light scattering; DOTAP is 1,2-dioleoyl-3-trimethylammonium-propane; DPPC is 1,2-dipalmitoyl-sn-glycero-3-phosphocholine; EV is extracellular vesicle; eEV is engineering extracellular vesicle; FBS is fetal bovine serum; GFP is green fluorescence protein; GMFI is normalized geometric mean fluorescence intensity; hMSCs is human mesenchymal stem cells; miRNA is microRNA; NTA is nanoparticle tracking analysis; OD is optical density; PBS is phosphate buffered saline; PEG is polyethylene glycol; POPC is 1-Palmitoyl-2-oleoyl-sn-glycero-3-phosphocholine; POPG is 1-Palmitoyl-2-oleoyl-sn-glycero-3-phosphoglycerol; PVDF is polyvinylidene fluoride; RNAi is RNA interference; siRNA is short interfering RNA; shRNA is short hairpin RNA; SPV is sulfo-phospho-vanillin; and TEM is transmission electron microscope.

In one embodiment of the present invention there is provided an engineered extracellular vesicle (eEV), comprising an extracellular vesicle isolated from a biological cell with at least one lipid incorporated into the membrane thereof.

In this embodiment the lipid may be a synthetic lipid or an exogenous lipid/non-native lipid or a combination thereof. Particularly, the synthetic lipid May 1-palmitoyl-2-oleoyl-sn-glycero-3-phosphocholine (POPC), 1-palmitoyl-2-oleoyl-sn-glycero-3-phosphoglycerol (POPG), dipalmitoylphosphatidylcholine (DPPC), or, 1,2-dipalmitoyl-sn-glycero-3-phosphocholine (DOTAP) or a combination thereof. Also, the biological cell may be associated with a pathophysiological condition. In one aspect the pathophysiological condition may be a cancer. In another aspect the biological cell may be a primary mesenchymal stem cell, an embryonic kidney cell, an embryonic fibroblast cell, an alveolar basal epithelial cell, or a monocytic cell or an immortalized cell-line thereof.

In another embodiment of the present invention there is provided a method for preparing an engineered extracellular vesicle, comprising the steps of culturing the biological cell as described supra in vitro in a culture medium; isolating the extracellular vesicles from the biological cells; and extruding the isolated extracellular vesicles with the at least one lipid to form the engineered extracellular vesicle.

In yet another embodiment of the present invention there is provided an extracellular vesicle delivery vehicle, comprising at least one lipid hybridized with a membrane of the extracellular vesicle; a nucleic acid loaded within a core of the extracellular vesicle; a multi-layered polyelectrolyte coating deposited around the extracellular vesicle; and a therapeutic drug complexed with the multi-layered polyelectrolyte coating.

In this embodiment the lipid may be a synthetic lipid comprising 1-palmitoyl-2-oleoyl-sn-glycero-3-phosphocholine (POPC), 1-palmitoyl-2-oleoyl-sn-glycero-3-phosphoglycerol (POPG), dipalmitoylphosphatidylcholine (DPPC), or, 1,2-dipalmitoyl-sn-glycero-3-phosphocholine (DOTAP) or a combination thereof. Also the nucleic acid may be a synthetic DNA, a naturally occurring DNA, a synthetic RNA, or a naturally occurring RNA, or fragments thereof. Particularly, the synthetic RNA or naturally occuring RNA may be a small-interfering RNA (siRNA) or a microRNA (miRNA). Also, the multi-layered polyelectrolyte coating may comprise alternating layers of oppositely charged polyelectrolytes. Particularly, the oppositely charged polyelectrolytes may be poly-L-lysine, polyacrylic acid or poly-β-amino ester or other anionic polyelectrolyte or cationic polyelectrolyte structured for oppositely charged complexation. In addition the therapeutic drug may be an anti-cancer drug. Particularly, the anti-cancer drug may be an aptamer, an antibody, a duobody or other therapeutic protein, or a small molecule drug.

In yet another embodiment of the present invention there is provided method for treating a pathophysiological condition in a subject in need of such treatment, comprising administering to the subject an amount of the extracellular vesicle delivery vehicle as described supra effective to at least decrease a population of cells associated with the pathophysiological condition. In this embodiment the pathophysiological condition may be a cancer.

In yet another embodiment of the present invention there is provided method for for co-delivering a nucleic acid and a therapeutic drug to a cell of interest, comprising contacting the cell of interest with the extracellular vesicle delivery vehicle as described supra. In this embodiment the cell of interest may be a cancer cell.

In yet another embodiment of the present invention there is provided a method for preparing an extracellular vesicle delivery vehicle, comprising the steps of culturing biological cells in vitro in a culture medium; isolating the extracellular vesicles from the biological cells; extruding the isolated extracellular vesicles with at least one lipid to form a lipid-hybridized extracellular vesicle; loading a nucleic acid into a core of the lipid-hybridized extracellular vesicle; depositing, sequentially, a first layer of a polycation to coat the lipid-hybridized extracellular vesicle, a second layer of a polyanion onto the first layer and a third layer of a cationic polymer ester onto the second layer; and complexing a therapeutic drug to the second layer to form the extracellular vesicle delivery vehicle.

In this embodiment the biological cell may be a cancer cell. Also in this embodiment the lipid may be a synthetic lipid comprising 1-palmitoyl-2-oleoyl-sn-glycero-3-phosphocholine (POPC), 1-palmitoyl-2-oleoyl-sn-glycero-3-phosphoglycerol (POPG), dipalmitoylphosphatidylcholine (DPPC), or, 1,2-dipalmitoyl-sn-glycero-3-phosphocholine (DOTAP) or a combination thereof. In addition the polycation may be poly(L-lysine), the polyanion may be poly(acrylic acid) and the cationic polymer ester may be poly(β-amino ester). Furthermore the nucleic acid may be a synthetic DNA, a naturally occurring DNA, a synthetic RNA, or a naturally occurring RNA, or fragments thereof. Further still the therapeutic drug may be an anti-cancer drug comprising an aptamer, an antibody, a duobody or other therapeutic protein, or a small molecule drug.

Provided herein are engineered extracellular vesicles and layer-by-layer (LbL)-coated engineered extracellular vesicles produced therefrom which are utilized as a delivery platform. The LbL-coated eEV are structured for targeted co-delivery of a therapeutic drug, for example, but not limited to, an anti-cancer drug, and a nucleic acid, for example, a synthetic or naturally occurring DNA or RNA or fragments thereof. The targeted co-delivery of the therapeutic drug and the nucleic acid via the (LbL)-coated engineered extracellular vesicles are used in methods for treating a pathophysiological condition. Moreover, such targeted co-delivery may decrease the likelihood of drug resistance and degradation of the nucleic acids by the actions of nucleases.

Thus, also provided are methods for preparing engineered extracellular vesicles (eEV) and LbL-coated engineered extracellular vesicles (LbL-eEV). Generally, extracellular vesicles are isolated from a biological cell of interest, for example, but not limited to, a cancer cell. The membranes of the extracellular vesicles are hybridized or modified with at least one synthetic lipid, for example, a synthetic hydrated lipid, such as by extrusion, to produce a modified or hybrid engineered extracellular vesicles. Examples of suitable synthetic lipids are, but not limited to, 1-Palmitoyl-2-oleoyl-sn-glycero-3-phosphocholine (POPC), 1-Palmitoyl-2-oleoyl-sn-glycero-3-phosphoglycerol (POPG), dipalmitoylphosphatidylcholine (DPPC), or, 1,2-dipalmitoyl-sn-glycero-3-phosphocholine (DOTAP).

Generally, to produce the layer-by-layer engineered extracellular vesicles (LbL-eEV) an exogenous nucleic acid is loaded within the core of the engineered extracellular vesicle and the surface of the extracellular vesicle is coated with a polyelectrolyte multilayer in which the layers alternate charge. The therapeutic drug is then loaded by complexing with one of the polyelectrolyte layers. The process of ionic complexation may be in any order and begin or end with a cationic or anionic entity using a charge-modified or non-charge modified cell by any means. The LbL-coated engineered extracellular vesicles may be prepared in the presence of a sugar, for example, trehalose, to mitigate aggregation of the engineered vesicles.

In a non-limiting example, a synthetic siRNA or naturally occurring siRNA is loaded via electroporation into the core of engineered EVs (eEVs) isolated from a cancer cell, such as, but not limited to, lung cancer cells, to decrease the siRNA exposure to nucleases. In a layer-by-layer (LbL) assembly a first layer of a polycation, such as poly(L-lysine) (PLL), is deposited on the weakly anionic extracellular vesicle surface to coat the same. Next, a second layer of a polyanion, such as poly(acrylic acid) (PAA), is deposited on the first layer to enable loading of the therapeutic drug, such as an anti-cancer drug, for example, but not limited to, doxorubicin (DOX). A third layer of a cationic polymer ester, such as poly(β-amino ester) (PBAE), is deposited on the second layer to facilitate siRNA delivery.

In addition provided herein is a method of treating a pathophysiological condition. Targeted co-delivery of both a therapeutic drug and a nucleic acid via the LbL-eEVs enables both drug-induced cell death and nucleic acid mediated gene silencing. Examples of a pathophysiological condition are cancers, such as, but not limited to, lung cancer. The therapeutic drug may be an anti-cancer drug, such as, but not limited to, doxorubicin, a protein, or a small molecule drug. The nucleic acid may be a DNA or an RNA, such as a small-interfering RNA (siRNA) or a micro-RNA (miRNA). The co-delivery of the therapeutic drug and nucleic acid to a subject is effective to treat the pathophysiological condition, for example, by inducing cell death or decreasing cell or tumor growth.

The following examples are given for the purpose of illustrating various embodiments of the invention and are not meant to limit the present invention in any fashion.

### EXAMPLE 1

### Materials and Methods

### Materials

[symbol] ,2-dioleoyl-3-trimethylammonium-propane (chloride salt) (DOTAP) (#890890C-25 mg, Avanti Polar Lipids, Inc.); 1-Palmitoyl-2-oleoyl-sn-glycero-3-phosphocholine (POPC) (#COATSOME® MC-6081, NOF America Corporation Ltd.); 1,2-dipalmitoyl-sn-glycero-3-phosphocholine (DPPC) (#850355C, Avanti Polar Lipids, Inc.); 1-Palmitoyl-2-oleoyl-sn-glycero-3-phosphoglycerol, sodium salt (POPG-Na) (#COATSOME® MG-6081LS, NOF America Corporation Ltd.); 0.4 cm electroporation cuvette (#1652088, Biorad); 400 mesh Formvar/carbon-coated copper grids (#FCF400-CU-50, Electron Microscopy Sciences); 6×DNA loading Dye (#R0611, Thermo Fisher Scientific); anti-CD63-eFluor 660 antibodies (#50 112 4280, Thermo Fisher Scientific); CellTiter 96® Aqueous Cell Proliferation assay (Promega G5421); cholesterol (#ICN10138201, MP Biomedicals); chloroform (molecular biology grade, Thermo Fisher Scientific); anhydrous D-trehalose (#AC309870250, ACROS); bovine serum albumin (BP1600-100, Thermo Fisher Scientific); Deep Red Plasma Membrane Stain Cell Mask (Life Technologies, #C10046); Dulbecco's Modified Eagle's Media (DMEM, #10-014-CV, Corning); Dulbecco's Modified Eagle's High Glucose Media (#10-013-CV, Corning); modified Eagle's media (EMEM, Lonza); dynamic light scattering (Zetasizer) cuvette (ZEN0040); fetal bovine serum (FBS) (#10437028, Gibco); GeneRuler 100 bp DNA ladder (#SM0241, Thermo Fisher Scientific); Glass coverslips (Thermofisher, #1254581); Hoechst33258 (Invitrogen, #H3569); glutaraldehyde (#16019, EMS); isopropanol (#AC412790010, ACROS); Lipofectamine RNAiMax (Invitrogen); Micro bicinchoninic acid (Micro BCA) protein assay kit (#23235, Thermo Fisher Scientific); methyl cellulose (#M6385, Sigma); non-essential amino acids (MEM-NEAA, #11140050, Gibco); O-Phosphoric acid (#A242500, Thermo Fisher Scientific); Opti-MEM I Reduced Serum Media (#31985062, Thermo Fisher Scientific); OptiPrep™ Density Gradient Medium (#D1556, Sigma); oxalic acid (#423152500, ACROS); paraformaldehyde (#28906, Thermo); penicillin (Invitrogen); penicillin-streptomycin (Invitrogen); phosphate buffered saline (PBS) (#10010023, Gibco); phosphotungstic acid (#P4006, Sigma); potassium chloride (#BP366500, Thermo Fisher Scientific); potassium phosphate dibasic (#BP363500, Thermo Fisher Scientific); TRIzol reagent (Invitrogen); Triton X-100 (Invitrogen); SYBR Safe DNA gel stain (#S33102, Invitrogen); trypsin-EDTA, 0.05% (wt/vol; Gibco); uranyl acetate (#NC0788109, Thermo Fisher Scientific); vanillin 99% (#AAA11169-22, Alfa Aesar); GlutaMax (Thermo Fisher Scientific); poly (acrylic acid) (Mw: 1800 g/mol, Sigma); poly(L-lysine) (Mw: 30000-70000 g/mol, MP Biomedical); and Quant-iT RiboGreen RNA assay kit (Thermo Fisher Scientific).

### Extracellular Vesicle Induction and Isolation

3T3 and A549 cells were grown with complete media until ˜100% confluent. The media was removed from the culturing flasks and the flasks were washed with phosphate-buffered saline (PBS) twice to remove any remaining media. After which, the cells were continued to be cultured in FBS-depleted media for 48 hr to promote the secretion of EVs. The conditioned media was collected and the EVs were isolated by serial centrifugation at 4° C. (32,33), using a TX150 rotor and ST8 centrifuge. The media was centrifuged at 300 g for 10 min., 2,000 g for 20 min., and 10,000 g for 30 min. The first, second, and last centrifugations were to remove intact cells, dead cells, and cell debris, respectively. Note that the pellet was discarded after each centrifugation. The supernatant media was then transferred to ultracentrifuge tubes and centrifuged at 125,000 g (Beckman Coulter centrifuge with TLA-55 rotor) for 70 min at 4° C. The pellet was resuspended in PBS, then filtered using a low protein binding polyvinylidene fluoride (PVDF) 0.22 μm filter. The isolated EVs were stored at a concentration of 10¹⁰ particles/ml at −20° C. To remove unwanted non-vesicular particles, OptiPrep™ density gradient solutions (Sigma, #D1556) were used to separate particles by their density (exosome: 1.1-1.19 g/ml) according to literature (32). In the small-scale preparation, no significant difference of protein: lipid ratios between the samples using ultracentrifugation and OptiPrep™ density gradient centrifugation were observed, however.

### Engineered Extracellular Vesicles via Extrusion

The lipids DOTAP, POPC, DPPC, and POPG are cationic, zwitterionic, zwitterionic, and anionic, respectively, are used to engineer the exosome-lipid hybridized membrane. The lipophilicity of lipids was calculated from open source LogP (34) prediction software, namely, Molinspiration (version: miLogP2.2 2005). The LogP values generated from the mentioned software are referred to as “miLogP” values and the transition temperatures are in Table 1.

TABLE 1
Lipophilicity and Transition Temperatures

| Lipid | miLogP | Transition Temp |
|---|---|---|
| DOTAP | 9.51 | <5° C. |
| POPC | 6.91 | −2° C. |
| DPPC | 6.38 | 41° C. |
| POPG | 8.95 | 2° C. |

The eEVs were fused using a serial extrusion technique. The lipid solutions were dissolved in chloroform (10 mM) according to literature. The organic solvent was evaporated in a vacuum chamber to yield a thin lipid film on the bottom of a glass vial. The lipid film was then hydrated by adding a PBS buffer. Prior to membrane extrusion, the lipid solutions were heated at a temperature above their phase transition temperature and vortexed until they were visually homogeneous. The extracellular vesicles were subsequently warmed to 37° C. The lipid:EV solutions were mixed at varying volumetric ratios (9:1, 4:1, 1:1); the lipid solution was at a concentration of 5 mM and the EV concentration was 1.5×10¹⁰ particles/ml (or 50 μg/ml of protein according to a Micro BCA assay. The mixtures were vortexed and sonicated for 2 min using a 120 Watt, 20 kHz sonicator (Fisher Scientific FB120) at 20% max amplitude to fully solvate the solution. Subsequently, the mixtures were serially extruded through pore sizes of 400 nm, 200 nm, and then 100 nm. For each extrusion procedure, the mixtures were push forward and backward manually more than 25 times according to the manufacturer's instruction (T&T Scientific).

### Layer-by-Layer Polyelectrolyte-eEV Complex (LbL-eEVs) Preparation

The LbL-eEVs were designed to have three polyelectrolyte layers, which were poly L-lysine (PLL, Mw: 30,000˜70,000 g/mol), poly(acrylic acid) (PAA, Mw: 1800 g/mol), and poly(β-amino ester)s (PBAE, Mw: 4001 g/mol). Exogenous siRNA was loaded into the eEVs via electroporation prior to the LbL deposition procedure. The LbL architecture was achieved by sequential deposition of oppositely charged polyelectrolytes. PLL was chosen because it is a widely used biocompatible and biodegradable cationic polypeptide (35). PAA is a highly anionic polyelectrolyte that has also been widely used for LbL assembly in the past (36). Additionally, small molecule drug doxorubicin (DOX) has been shown to complex with PAA via electrostatic interactions (37,38). Cationic PBAE was chosen as a final layer because of its high gene transfection efficiency and because it has been reported to improve intracellular delivery in previous research (39,40). The specific PBAE used for LbL coating was BR647, which is a bio-reducible and hydrophobic PBAE with a disulfide bond along the polymer backbone and was synthesized according to a previous protocol (41) The hydrolysis of the ester group on the BR647 polymer and the degradation of the disulfide bonds can be triggered by the reducing environment of the cytoplasm, promoting cargo release. Briefly, in the LbL process, ˜5×10¹¹ particles/ml of eEVs were mixed with a final concentration of 200 μg/ml PLL in 150 mM sodium acetate (NaAc, pH=5) and incubated in 37° C. for 1 hr. The mixture was then ultracentrifuged at 125000 g for 30 min to pellet polyelectrolyte-coated eEVs. The supernatant was replaced with 150 mM NaAc for washing, and the ultracentrifugation/wash procedure was repeated twice to remove excess polyelectrolytes. Subsequent layers were deposited by the same procedure with 30 min of incubation, followed by the same washing procedure. 2^{nd} layer: PAA (0.5 mg/ml in 150 mM NaAc), drug loading: DOX (0.3 or 0.6 mg/ml in 150 mM NaAc) and 3^{rd} layer: PBAE-BR647 (2.5 mg/ml in 25 mM NaAc) were added sequentially for LbL deposition. To reduce acidity, 25 mM NaAc was used instead of 150 mM NaAc for the final PBAE coating. The final layer of PBAE-BR647 was not washed according to the protocol of Bishop et al. (42).

### Physicochemical Quantification of EVs From Cells and eEVs

Membrane protein quantification assay: The total quantity of protein within EVs was quantified via a Micro BCA Protein Assay Kit, according to the manufacturer's instructions. Briefly, a set of protein standards was prepared within the linear working range of 2-40 μg/ml. EV samples with different concentration were mixed with working solution in a 96 well microplate and incubated at 37° C. for 2 hr. Absorbance in each well was measured using a plate reader (Cytation 5, BioTek Instruments, Inc.) at 562 nm.

Flow cytometry—Characterization of EV surface marker and purity: EVs obtained after differential centrifugation and filtration were then analyzed by flow cytometry for the presence of exosome marker CD63 (43,44). Initially, EVs were diluted to the concentration of 5000 ng of affiliated protein or a total 1010 particles in 50 μl of PBS solution. The solutions were subsequently mixed with 0.125 μg (1.25 μl) of anti-CD63-eFluor 660 antibodies to a final volume of 100 μl of PBS with 0.2% bovine serum albumin (BSA) blocking solution. The mixture was incubated at room temperature for 30 min. in the dark before conducting flow cytometry analysis. Flow cytometry was accomplished using a BD Accuri™ C6 Cytometer at a flow rate of 11 μl/min. Auto-fluorescence was quantified using the samples in the absence of the anti-CD63-conjugated eFluor 660 antibody. POPC, which has no CD63 surface marker expression, was used to identify the background fluorescence via flow cytometry for gating purposes. To characterize the percentage of CD63 expressing vesicles or the percentage of EVs within the solution, the percentage of eFluor 660 fluorescence was quantified using the FL4-H channel.

Nanoparticle tracking analysis—size, concentration and production yield: The size and concentration of exosomes were characterized by nanoparticle tracking analysis (NTA) (Malvern NanoSight LM10, Amesbury, United Kingdom). An appropriate working concentration in the measurable range of 108 particles/ml was used to determine the original concentration of EVs. The EV samples with serial diluted concentrations (in PBS) were injected in the NTA sample chamber using sterile syringes. Data for each sample were collected for 60 s at room temperature and analyzed using NanoSight NTA 3.2 software. Three individual measurements of each condition were performed immediately after the sample was injected into the chamber. The error bars shown are standard deviations of the mean size and the original concentration was calculated using a dilution factor. A dilution factor was necessary to be within the measurable concentration range for NTA (10⁷⁻⁹ particles/ml). The production yield of EVs was further normalized to the cell number per flask (or dish) at ˜100% confluency, counted by hemocytometer. Likewisely, an appropriate working concentration of eEVs in the NTA measurable range of 10⁸ particles/ml was prepared to determine the size and concentration of eEVs. The concentration of eEVs were normalized to the concentration of native EVs to quantify the production yield, in terms of the fold increase of eEVs.

Transmission electron microscopy—morphology: A pellet of EVs or eEVs (˜10⁸ particles) was resuspended in 50 μl of PBS and stained on Formvar/carbon-coated copper grid for microscopy imaging purposes following the protocol in previous literature (45). Details of the steps for sample staining are depicted in Supporting Information. A drop of 5-10 μl of EV suspension was put on clean Parafilm. The sample grids were then allowed to vacuum dry overnight and observed via TEM (JEOL 1200EX) under 100 kV of energy and 100,000× to 150,000× magnification.

Zeta potential measurements: To obtain information about the stability of the vesicles in terms of particle aggregation and flocculation, the zeta potentials of the vesicles were evaluated measured in PBS and water. The zeta potential of vesicles was measured using a Zetasizer (NanoZS) from Malvern Instruments with a detection angle of 173° and laser wavelength of 633 nm (cuvette: ZEN0040). 10 μg/ml (affiliated protein concentration) of EV and 10⁹-10¹⁰ particles/ml of eEVs were used for the measurements.

### Quantifying Native EV Fraction Within eEVs for Validating Membrane Incorporation

Membrane composition quantification assay: To validate the membrane incorporation of lipids to extracellular vesicles, we conducted membrane composition analysis to quantitatively evaluate the efficiency of membrane fusion. It was demonstrated that the protein to lipid ratios characteristic could be a consistent parameter to characterize extracellular vesicles populations (46). Therefore, we quantify the EV fraction within eEVs by quantifying the protein (which is EV-derived) and the lipid content (which is both EV- and synthetic lipid-derived) within. The protein quantification method (Micro BCA) was described previously in Membrane protein quantification assay. A sulfo-phospho-vanillin (SPV) assay was used following previous protocol (46). Total protein to total lipid ratios were further calculated to determine the fusion efficiency of exosomal membrane. To confirm the lipid molecules presenting in the samples did not interfere with the total protein determination in terms of causing false-positive protein results, the pure lipid solution was tested for their assay interference with the protein determination, followed by quantification of the total protein to lipid ratios for pure lipid solution.

Flow cytometry-based eEV/EV ratio quantification assay: To quantify the native EV portion within the eEVs, anti-CD63 conjugated eFluor 660 antibody was used as a labeling marker for EVs. The same procedure for sample preparation was conducted as described in Flow cytometry—Characterization of EV surface marker and purity. The amount of eFluor 660 fluorescence was quantified using the geometric mean of FL4-A of the vesicle population, which is commensurate with the amount of CD63 per exosome. The fraction of EV incorporation with synthetic lipids were calculated using the normalized geometric mean values, according to the following equation (note that the EV fraction in eEVs=1−Synthetic lipid fraction):

$$EV fraction in eEVs = \frac{{GM}_{eEV}}{{GM}_{background}} / \frac{{GM}_{EV}}{{GM}_{background}} = {GM}_{eEV} / {GM}_{EV} \qquad ( eq . 1 )$$

Auto-fluorescence or background fluorescence was quantified using samples in the absence of the anti-CD63-eFluor 660 antibody. POPC was used as a stained negative control to compare with native EVs. Isotype igG1 was also pre-tested to get rid of the nonspecific binding concern of antibodies. Note that the above equation will result in values ranging from 0 to 1, where 0 is 100% synthetic lipids and 1 is 100% native EV.

### EV and eEV siRNA Loading

siRNA loading method: siRNA was loaded within EVs and eEVs via electroporation using a modified protocol as was previously described (47). Briefly, electroporation mixture was prepared in a concentration of 100 μg/ml EV (3×10¹⁰ particles/ml) or 10¹⁰-10¹¹ particles/ml of eEV containing 1 ng/μl siRNA. Note the mass of EVs is represented by the affiliated protein amount as quantified by the Micro BCA assay. Opti-MEM or a hypotonic electroporation buffer (1.15 mM K₂HPO₄; pH 7.2; 25 mM KCl, 21% OptiPrep according to the protocol (47,48) were used as the sample solution. Hypotonic buffer was proposed to drive a faster water uptake across vesicle membrane by an imposed osmotic gradient, which facilitates the uptake of genes, resulting in an increase of the transfection efficiency (49-51). Sample volumes of 100 μl were used for electroporation. The siRNA/vesicle mixtures were then transferred to 0.4 cm electroporation cuvettes (#1652088, Biorad) and electroporated using a Bio-Rad Gene Pulser II system, using the following conditions: 400 V, a capacitance of 125 μF, and an exponential pulse induction process. 2 pulses were applied to increase the nucleic acid incorporation or entrapment within the EVs and eEVs, as described previously (52). After electroporation, samples were kept on ice for 1 hr to allow for membrane recovery prior to further experiments.

Loaded siRNA content quantification: To quantify the amount of siRNA loaded within EVs/eEVs post-electroporation, the samples were ultracentrifuged at 125000 g for 70 min. twice to remove free siRNA in solution. The supernatant was discarded after ultracentrifugation and fresh PBS was replenished to each sample as an ultracentrifuge-wash cycle to completely remove loosely bound, free siRNA from EVs/eEVs. As a first step in evaluating the capability of exogenous cargo loading, the actual siRNA amount loaded into the vesicles was quantified. To isolate siRNA from vesicles, a modified TRIzol RNA isolation protocol was used according to previously published literature (53). After siRNA isolation, Quant-iT RiboGreen RNA fluorescent dye (preferentially fluoresces in the presence of RNA (when the nucleic acid is <500 bp)) was used for the quantification of siRNA, following the manufacturer's instruction. siRNA at varying concentrations were prepared and measured to generate an siRNA calibration curve. The amount endogenous RNA within the exosomes was quantfied, which merely measured at 0.8±0.3 ng of nucleic acid from 10⁹ (1E9) exosomes. Compared to the exogenous loading amounts of siRNA (100 ng), it is only ˜1% of the total amount.

As a control between different lipid-doped systems, a concentration of ˜E10 particles per formulation was carried out under the same vesicle densities for accurate comparison of electroporation efficiency. The loading efficiency (left y-axis) is determined by equation 2 which represents as siRNA copies per vesicle:

$$\frac{Numbers of siRNA in vesicles}{Numbers of vesicles} \times 100 \% / \qquad ( eq . 2 )$$

The encapsulation efficiency (right y-axis) is determined by equation 3:

$$\frac{Mass of siRNA in vesicles}{Mass of the feeding siRNA} \times 100 \% . \qquad ( eq . 3 )$$

### Mitigation of Cytotoxicity and Aggregation

Cell viability assay: The relative metabolic activity of the samples was evaluated using the CellTiter 96® Aqueous Cell Proliferation assay (Promega) (MTS assay) to assess the viability or cytotoxicity levels. Prior to MTS treatment, siRNA-loaded eEVs were incubated with cultured A549 cells at 60-70% confluency for 2 hr. After 2 hr incubation, cells were washed to remove excess eEVs and replaced with fresh media for further cell incubation. The MTS assays were conducted after 24 hr of siRNA treatment, following the manufacturer's instruction. Recent animal studies of EVs reported that a dose of 10⁹-10¹¹ EVs (with 1-500 μg siRNA) per 10⁵-10⁶ tumor cells (initial cell number) is typically required to achieve therapeutic effects when it is administered every day or every other day (47, 54-56). In regard to the cell viability assay and aggregation studies, we delivered a single dose of 10⁹-10¹⁰ eEVs with 100-400 ng siRNA per 104 cells.

Aggregation determined by microscopic image analyses and spectroscopy assays: In addition to NTA and DLS measurements for particle number and size quantification, the degree of aggregation was quantified by light a microscopy imaging method (57) using Cytation 5 for electroporation-induced aggregates. Particle diameter and number were quantified by Cytation 5's imaging analysis software. To distinguish between background and particles, the bright field intensity was set at 5000 a.u. and the particle size range from 0.5 to 100 μm were set as thresholds. 5 spots per well were imaged and were analyzed. Additionally, spectroscopic assays were employed according to previous studies (58-60) to determine the turbidity (i.e. the absorbance) of the solution. Absorbance spectra were quantified between 230 nm and 998 nm using a spectrometer (Cytation 5). The reading was recorded immediately after loading samples into a 96 well UV transparent plate. The maximum optical density (OD) in the spectrum at 230 nm was used to compare between samples.

Mitigation of aggregation and cytotoxicity via electroporation parameters: To assess the electroporation media effects on electroporation-induced aggregation, Opti-MEM, Opti-MEM+EDTA, 50 mM trehalose, and hypotonic electroporation buffer were prepared. In regard to the condition of Opti-MEM+EDTA, 5 mM EDTA was added to the electroporated mixture immediately after electroporation following the protocol from Lamichhane, et al. who reported similar effects can be obtained with EDTA addition either before or after electroporation (52). 50 mM trehalose, shown by Hood et al. as a membrane stabilizer to ameliorate the electroporation induced-aggregation (61), was prepared accordingly. Commonly used hypotonic electroporation buffer (47, 62) was also prepared as mentioned the siRNA loading section.

### Quantification of RNAi Knockdown and eEV Targetability

Quantification of siRNA knockdown in A549 cells: To evaluate the knockdown efficiency of EV-mediated siRNA delivery within the constitutively expressing-GFP-A549 cells over time, GFP fluorescence was measured and quantified using a plate reader (Cytation 5) every day for 12 days (34). To ensure the decrease of green fluorescence intensity is due to the RNA interference of GFP-siRNA in the cells and not caused by the cytotoxicity of the delivery system, each sample type or condition was delivered with scrambled siRNA as a control. The knockdown efficiency was calculated using the below equation:

$$Knockdown \% = 100 \times ( 1 - ( {F}_{si} - {F}_{bg} ) / {F}_{osi} \times {F}_{osc} / ( {F}_{sc} - {F}_{bg} ) ) \qquad eq . ( 4 )$$

where Fₛᵢ is the fluorescence of the well using GFP-siRNA, F_{sc} is the fluorescence of the well using corresponding scrambled siRNA, F_{bg} is the fluorescence background of the media without cells, Fₒₛᵢ is the initial fluorescence of the well just prior to delivery for the GFP-siRNA formulations and F_{osc} is the initial fluorescence of the well prior to delivery of the corresponding scrambled siRNA control group. Commercial Lipofectamine® RNAiMax formulation was used as a positive control to compare the knockdown efficiency between samples. The formulation was prepared according to the manufacturer's instruction. Briefly, 0.3 μl of Lipofectamine® RNAiMax was used to deliver 1 pmol of siRNA. The siRNA-Lipofectamine® RNAiMax complexes were mixed in Opti-MEM® media and incubated at room temperature for 10 min. before delivery to the cells. For the in vitro siRNA delivery and knockdown assessment, each sample mixture was delivered to the cells for 2 hr. After 2 hr, the sample solutions were replaced by the typical culturing media (with serum) for continual measurements. Area under the curve (knockdown efficiency vs time) was calculated to determine the difference in duration using GraphPad Prism 7 software.

Uptake experiments in A549 and CCL-210 cells: 2.5×10⁴ cells were seeded on glass coverslips 24 hours prior to imaging and quantification. Deep Red Plasma Membrane Stain Cell Mask was used to label EVs/eEVs following the protocol modified from previous literature. Briefly, EVs/eEVs were incubated with CellMask (1:10000 dilution) in PBS for 5 min. at 37° C. and then wash-centrifuge three times to remove free Deep Red dyes. Deep Red dye labeled-EVs/eEVs were subsequently added to the cells and incubated for 2 hours at 37° C. All cells were then fixed with 4% paraformaldehyde and counterstained with 5 μg/ml of Hoechst 33258 for nuclei visualization. Cell samples were imaged using a confocal microscope (Olympus FV1000). All the images were analyzed with Image J software. To further quantify the uptake efficiency in the cells, cells were washed with PBS, trypsinized and assayed using a BD Accuri™ C6 Cytometer at a 14 μl/min of flow rate. 10000 events were collected per sample with triplicates. The signal of Deep Red fluorescence was quantified using the geometric mean of FL4-A of the cell population, which is commensurate with the amount of EV/eEV uptake per cell. To eliminate any cell line-specific difference in the signal background, the following equation was used for calculation:

$$Uptake efficiency in cells = \frac{{GM}_{EV or eEV in A 549 or CCL 210}}{{GM}_{untreated cells ( A 549 or CCL 210 )}} / \frac{{GM}_{eEV in A 549}}{{GM}_{untreated A 549}} . \qquad eq . ( 5 )$$

Untreated A549 and CCL-210 cells were measured as a negative control. The uptake of POPC-EV in the absence of Deep Red dye staining in A549 and CCL-210 was also examined to confirm no auto-fluorescence interference from the vesicles. It is important to note that the excessive lipophilic dye staining with exosomes may cause non-specific binding to the cell membranes, resulting in false-positive signals or the change of exosome uptake pathways. The (dye only) control which was similarly centrifuged serially and assayed via flow cytometry indicated the background fluorescence was only 1.4±0.3% and 4.2±0.9% of the A549 and CCL210 cells' samples, respectively.

### Statistics

Data are presented as the mean±the standard deviation (SD). All experiments were conducted with triplicates (Exception are indicated specifically as below). Statistical data analyses were performed using GraphPad Prism 7 software. The α value was set at 0.05, where *, **, ***, **** represent p-values<0.05, <0.01, <0.001, and <0.0001 respectively. The following statistical tests were conducted: Unpaired, two-tailed Student's t-test, One-way ANOVA, followed by Dunnett's multiple comparison test (post-hoc), and One-way ANOVA, followed by Tukey's multiple comparison test (post-hoc).

### Cell Culture

Green fluorescent protein (GFP) expressing A549 lung adenocarcinoma cells were purchased from Cell Biolab, Inc. (#AKR-209) and further sorted by flow cytometry to obtain a higher percentage of GFP expressing A549 cell population as reported in the previous studies (63). A549 lung cancer cells (P5-P15) were cultured in DMEM high glucose (4.5 g/ml glucose) media supplemented with 10% v/v fetal bovine serum (FBS), 1% v/v 0.1 mM non-essential amino acids (MEM-NEAA), and 1% v/v 100 μg/ml penicillin-streptomycin. CCL-210 cells were cultured in modified Eagle's media (EMEM, Lonza) supplemented with 10% v/v FBS, 1% v/v GlutaMax, and 1% v/v 100 μg/ml penicillin-streptomycin. Both cell types were grown in an incubator using cell culture conditions of 37° C. and 5% CO₂.

### SiRNA Loading in eEVs, siRNA Retention and DOX Quantification in LbL eEVs

siRNA loading method: Herein, anti-GFP siRNA was loaded within zwitterionic POPC-doped eEVs via electroporation (Bio-Rad Gene Pulser II system) using a protocol previously reported. Briefly, an electroporation mixture was prepared in a concentration of ˜7.5×10¹¹ particles/ml of eEVs containing 1 ng/μl siRNA. Opti-MEM, 50 mM trehalose or a hypotonic electroporation buffer (1.15 mM K₂HPO₄; pH 7.2; 25 mM KCl, 21% OptiPrep) (47,48) were used as sample buffer. For siRNA-loaded LbL-eEV samples, polymer deposition processes were conducted after the electroporation procedure.

Loaded siRNA content quantification: To quantify the amount of siRNA within the LbL-eEVs post-assembly, the actual siRNA amount retained in the vesicles was isolated. A modified TRIzol RNA isolation protocol was used according to previously published literature (53). Purified siRNA was subsequently quantified by Quant-iT RiboGreen RNA assay kit, following the manufacturer's instruction.

Loaded DOX content quantification: Small molecule DOX was loaded to LbL-eEV after PAA complexation during LbL assembly. DOX loaded LbL-eEVs were pelleted down by centrifugation and resuspended in DMSO/PBS (3:7 volume ratio) solution. The amount of incorporated DOX in the LbL-eEVs was determined by measuring the fluorescent absorbance (Excitation: 485 nm/Emission: 590 nm) of DOX using a Cytation 5 spectrophotometer. A linear calibration curve with DOX concentrations in the range of 0-12.5 μg/ml were used to obtain unknown DOX loading amount.

### Cellular Uptake Studies by Confocal Microscopy and Flow Cytometry

CellMask™ DeepRed plasma membrane stain was used to label the membrane of LbL-eEVs following a protocol modified from previous literature (64). Same procedures of cell seeding, sample staining, and imaging were done as described in the previous protocol (63). For confocal microscopy imaging (Olympus FV1000), Hoechst 33258 was co-stained for cellular nuclei visualization. To further quantify the uptake efficiency of vesicles in the cells, the Deep Red fluorescence was detected by flow cytometry (geometric mean of FL4-A channel), and the following equation was used for calculation:

$$Uptake efficiency = \frac{{GM}_{LbL - eEV in A 549 or CCL 210}}{{GM}_{untreated cells ( A 549 or CCL 210 )}} / \frac{{GM}_{eEV in A 549}}{{GM}_{untreated A 549}} . \qquad eq . ( 6 )$$

To quantify the uptake efficiency of cargo following intracellular delivery, cells were treated with LbL-eEVs loaded with DOX/Cy3-siRNA. The FL2-A channel was used for the detection of DOX and siRNA. For siRNA uptake quantification, Cy3-labeled siRNA was prepared by Label IT siRNA Tracker Intracellular Localization kit (Mirus) according to the manufacturer's instruction and diluted to a concentration of 100 nucleotides/dye prior to loading into eEVs. The cells were incubated with 3×10¹² particles/ml of LbL-eEVs containing Cy3-siRNA. To compare the intracellular delivery efficiency, Lipofectamine reagent mixed with 500 ng/ml (50 ng in 100 μl) Cy3-siRNA was used as a positive control group. On the other hand, for DOX quantification, cells were treated with free unencapsulated DOX at a concentration of 0.488 μg/ml, which was equivalent to the amount of DOX within the eEV/LbL-eEVs, as a positive control group. The metabolic activity of the cells was not affected at this DOX concentration within 2 hours of incubation. DOX loaded poly(lactic acid-co-glycolic acid) nanoparticles (PLGA NP) were prepared as a comparison group using oil-in-water nanoprecipitation followed by solvent evaporation according to the protocol from Betancourt et al. (65). PLGA (50/50, 50 kDa) with ester end groups was used for this nanoparticle synthesis. The PLGA NPs had a Z-average diameter of 127.4±2.9 nm and a DOX loading efficiency of 6.3%.

### In Vitro Evaluation: Anti-Cancer Efficacy

siRNA knockdown efficiency: To evaluate the knockdown efficiency of LbL-EV mediated siRNA delivery within GFP-expressing A549 cells, GFP fluorescence was measured and quantified using the same apparatus (Cytation 5) every day as previously described (34, 63). The knockdown efficiency of siRNA-GFP was calculated by normalizing each sample type/condition to each individual negative control treated with scrambled siRNA. Commercial Lipofectamine® RNAiMax was used as a positive control to compare the knockdown efficiency between samples. Area under the curve (knockdown efficiency vs time) was calculated by GraphPad Prism 7 software to determine the overall knockdown efficiency of each condition. In this study, we delivered a single dose of 10¹⁰-10¹¹ LbL-eEVs with 100-400 ng siRNA per 10⁴ cells in 100 μl.

DOX cancer killing efficiency: The effects of DOX-loaded vesicles on A549/CCL210 cells were done by CellTiter 96® Aqueous Cell Proliferation assay (Promega) (MTS assay) to assess relative metabolic activity of the cells. Prior to MTS treatment, DOX-loaded vesicles were incubated with cultured A549/CCL210 cells at 60-70% confluency for 3 days at varying doses. The MTS assays were conducted after incubation for 3 days, following the manufacturer's instructions. Untreated A549 or CCL210 cells were used as positive control groups (assuming 100% metabolic activity) for normalization. Free DOX (unencapsulated) and DOX-loaded PLGA NPs were prepared as comparison groups. To obtain dose-response curves, metabolic activity was plotted versus the amount of DOX administered, which was determined based on spectrophotometric analysis of eEV/LbL-eEV/PLGA NPs. The sigmoidal dose-response curves were fitted with Hill's equation using GraphPad software:

$$Y = Min + \frac{( Max - Min )}{( 1 + {( IC 50 / x )}^{Hill Slope} )} \qquad eq . ( 7 )$$

where Max is the Y value at the top plateau and Min is the Y value at the bottom plateau. IC50 (inhibitory concentration, 50%) is the X value when the response is halfway between Min and Max.

### Co-Delivery of siRNA and DOX

This experiment followed the same procedure as was used for siRNA and DOX delivery. Briefly, 2.5×10⁴ GFP-expressing A549 cells were seeded onto 24-well plates for flow cytometry analysis 24 hr before the experiment, followed by incubation with various formulations for 2 hr at 37° C. After 2 hr, the samples were replaced by fresh media for continual measurements. After 3 days incubation, the cells were trypsinized and collected for analysis. Of note, a concentration of 1.5×10¹² particles/ml was chosen for these co-delivery experiments based on the knockdown results. At this concentration, with only 2 hrs of DOX delivery followed by 3 days of incubation, the cells were expected to maintain 60-80% of their metabolic activity at the time of flow cytometry analysis. The cells were stained with ethidium homodimer (EthD-1) prior to flow cytometry for dead cell quantification, and detection was performed on the FL3-A channel. While there is some potential for overlap from DOX, this channel is not optimal for DOX detection. Control groups of Triton-treated A549 and untreated A549 were used to set the quadratic gates.

### EXAMPLE 2

### Physicochemical Quantification of EVs From Cells

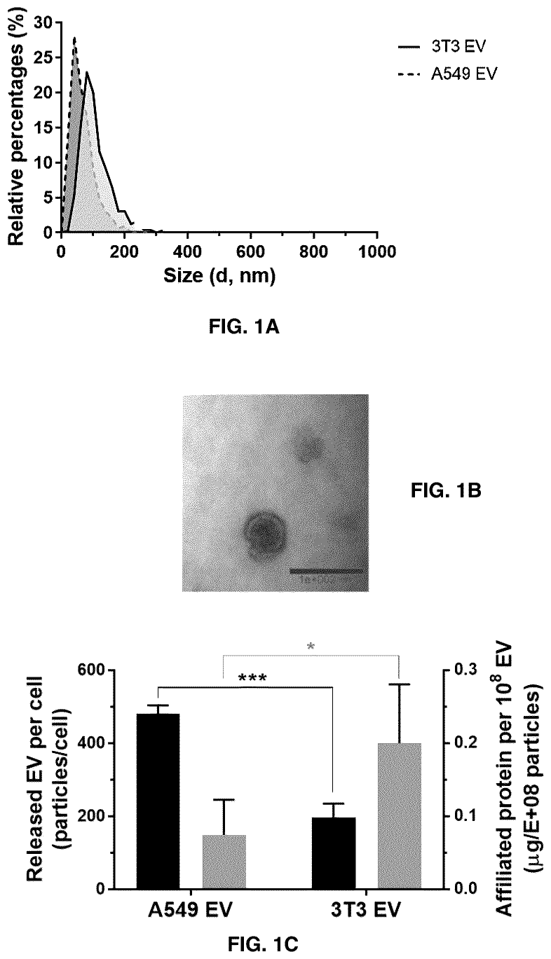

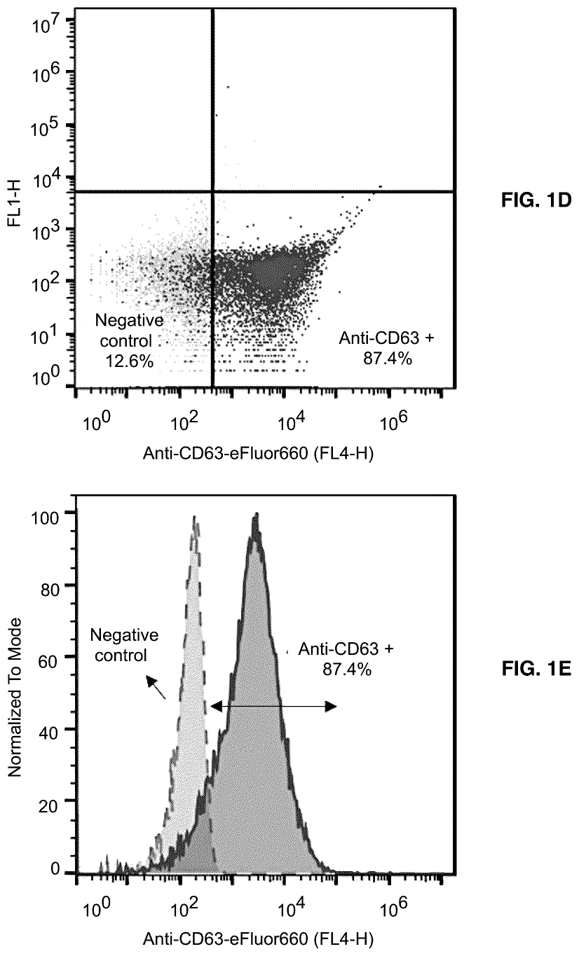

The isolated EV populations were initially characterized by transmission electron microscope (TEM), nanoparticle tracking analysis (NTA), total protein content and flow cytometry. Representative size distribution profiles of EVs revealed by NTA are shown in FIG. 1A. Average particle diameters of 124.3±14.7 nm and 90.1±18.6 nm were obtained for EVs derived from 3T3 (3T3 EVs) and A549 cells (A549 EVs), respectively. The EVs from both cell types had uniform and narrow size distributions. No larger particles above 500 nm were observed. TEM morphology (FIG. 1B) demonstrated a spherical shape of the vesicles. In terms of the production yield, 3T3 EVs on the order of (1.9±0.37)×10⁹ particles were obtained from 10⁷ cells, namely, 197±37 EVs per cell; whereas a higher amount of EVs were released per cell from A549 cells, on the order of (4.80±0.23)×10⁹ particles from 10⁷ cells (480±23 EVs per cell), as shown in FIG. 1C (left y-axis). Representative protein amounts of EVs quantified by Micro BCA assay are shown in FIG. 1C (right y-axis). Amounts of 0.20±0.08 μg protein and 0.08±0.05 μg protein within 10⁸ EV particles were quantified in 3T3 EVs and A549 EVs, respectively. These characterizations of EVs are similar to the production yields of U937 (human monocytic cells)- and HEK293T (human embryonic kidney cells)-derived exosomes reported from previous studies (56, 66).

As a model system for gene delivery, the yields of EV production from parental sources and the selectivity to the cells of interest need to be considered. This data demonstrated A549 cells had a 2.4-fold higher (***p<0.001) production yield of EVs compared to the EV yields of 3T3 cells. A549 cells were chosen as the source of EVs to generate batches of eEVs because of their relatively higher production yield and potential targetability of tumor derived-extracellular vesicles to tumor cells (67,68). Although human mesenchymal stem cells (hMSCs)-derived EVs are well-known as an efficient mass producer of EVs (69) (˜2100±300 released hMSC EVs/cell, ˜5-folds higher than A549 cells, based on the data), due to the limited expansion of hMSC culture (70) and favorable doubling time of A549 cells (>50 hours for hMSCs (71) whereas 22 hours for A549), A549 cells were used for the purpose of demonstrating the development of scalable eEVs. Isolated A549 EVs were then analyzed by flow cytometry for the presence of specific markers. As shown in FIG. 1D, fluorochrome-labeled anti-CD63 binding EVs and nonfluorescent isotype control showed discernible populations in the gate FL1 versus FL4 (anti-CD63-eFluor 660). Results showed CD63 is highly enriched in the isolated EV samples. 87.4% of the isolated samples were positive for the exosomal marker CD63 (FIG. 1E). Therefore, it was expected the majority of the EVs to be exosomes (CD63+), as opposed to microvesicles where CD63 was typically not detected (CD63−) (72).

### Physicochemical Quantification of eEVs From Cells

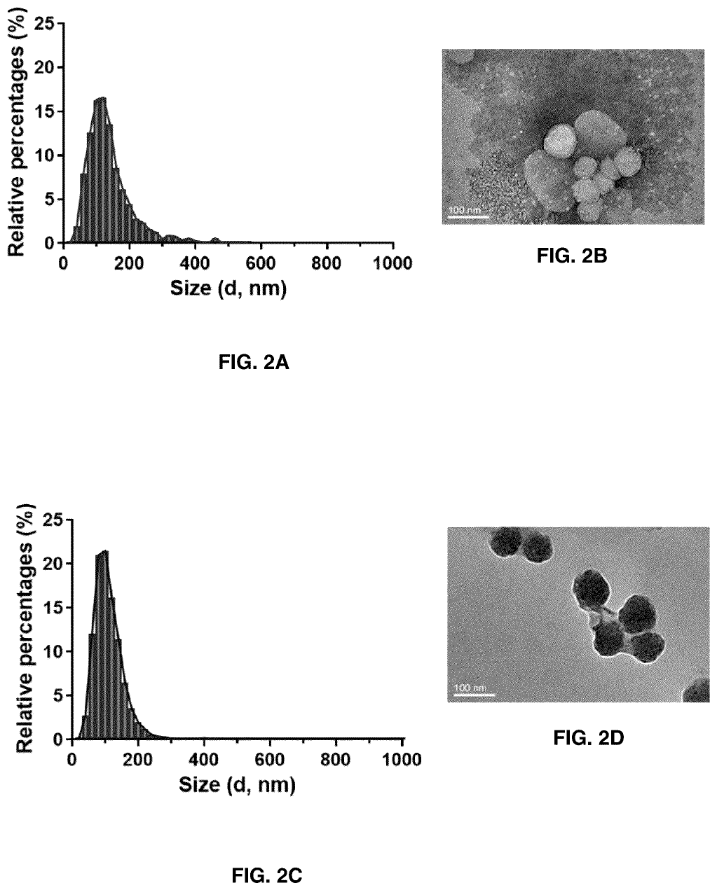

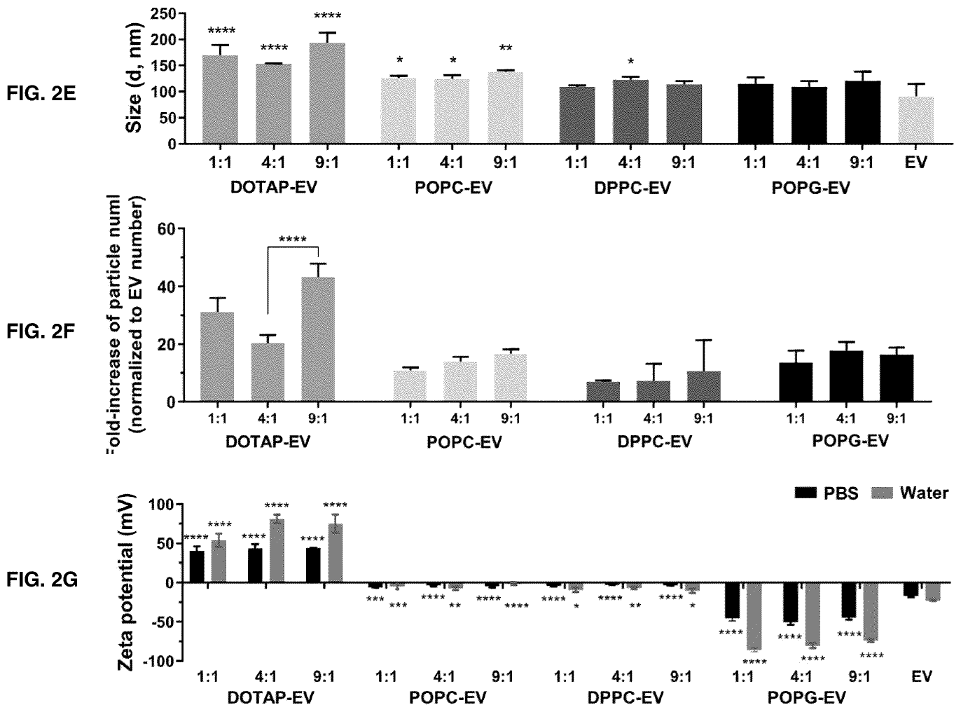

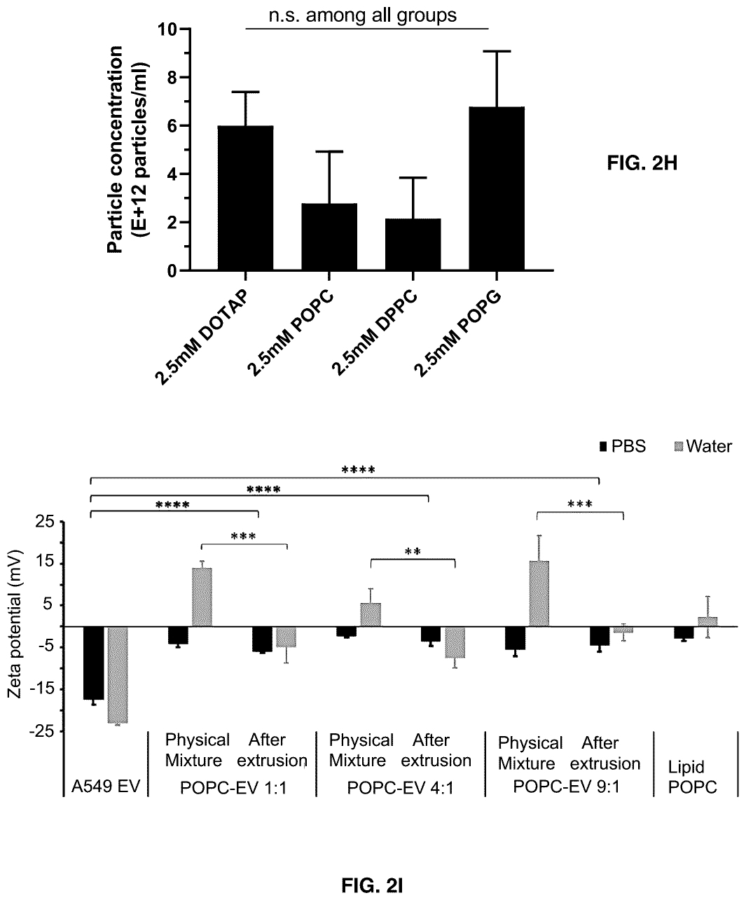

To generate eEVs, the isolated EVs were then fused with pre-hydrated lipids using a sonication and serial extrusion procedure. It was investigated whether different charges of lipids could be incorporated to native/naïve cell derived EVs. A library of lipids (DOTAP, POPC, DPPC and POPG) were therefore prepared for membrane hybridization. After producing lipid-doped eEVs, the morphology, size, mass production and zeta potential of eEVs were characterized. FIGS. 2A, 2C show the size distribution profiles of pre-hydrated POPC liposomes alone and POPC-doped eEVs, respectively: 138.8±9.1 nm and 126.3±4.0 nm (***p<0.001; statistically significant). The TEM images of FIGS. 2B, 2D depict the morphology of pre-hydrated POPC liposomes alone and intact structure of POPC-doped eEVs. While heterogeneous size of pre-hydrated liposomes was shown in FIG. 2A, a lamellar structure and uniform size of vesicles approximately 100 nm were demonstrated in FIG. 2C for eEVs. The TEM morphology confirmed the vesicle structure was not impaired by the sonication-extrusion method, indicating the extrusion apparatus is a suitable method for fabricating lipid-hybridized eEVs. A variety of lipid-doped eEVs at varying extruded ratios (from 1:1 to 9:1 of lipid to EV mixing ratio) were further characterized by performing the same sonication-extrusion processes, as shown in FIG. 2E. As expected, the average diameters of eEVs measured by NTA indicated that eEV formulations are much smaller than 200 nm size, thus these formulations would likely be able to take advantage of the enhanced permeability and retention effect (73). Among each individual lipid-doped eEVs, no statistical difference on the particle size for eEVs at varying extruded ratio was observed.

Several studies have endeavored to develop scalable techniques at the stages of EV generation and purification, such as: using a two compartment culture (Integra CELLine) (74); a microcarrier-based 3D culture (54); a hollow-fiber culture system (75); and a tangential flow filtration system for purification (54). Among these studies, a 7-fold to 40-fold increase in the production yield of EVs was achieved. In this study, instead of scaling up the production of EVs from the cells, it was attempted to mass produce the vesicles after EV isolation. By evaluating the quantity of eEVs using NTA measurements, it was observed a constantly higher production yields over various conditions (i.e. different lipid-doped eEVs and varying lipid:EV ratios) obtained by the sonication-extrusion technique compared to native EV production yields, as shown in FIG. 2F. On average, there was a 6-fold (for DPPC-EV 1:1 samples) to 43-fold (for DOTAP-EV 9:1 samples) increase on the overall amount of particle number upon the sonication-extrusion processes (FIG. 2F). While DOTAP-EV showed an average of 20-fold further increase of particle yields, POPC-EV, DPPC-EV and POPG-EV showed similar 10-fold increase values. Initial particle number of different lipid types in same molar concentration were also quantified in FIG. 2H. No statistical difference of particle number was shown among different types of lipids. These results indicated that the increase of particle number after sonication-extrusion technique most likely depends on the physical-chemical properties of lipids (i.e., charge), instead of the initial particle numbers of lipids. Overall, the production yields of eEVs generated post-EV isolation were about ˜8-fold higher than the number of EVs obtained from hMSCs using typical cell generation method, according to the results of hMSC EV from previous literature (54,69).

While others have integrated other synthetic materials (i.e. via freeze-thaw methods and PEG-induction methods) to already-isolated-EVs, such as liposomes and have proposed such systems as potential drug delivery carriers (76), the instant system highlights the ability to mass produce the number of vesicles, as opposed to multi-lamellar/hybridized EVs (77,78). Furthermore, this data of different lipid-doped eEVs demonstrate that the mass production can be tuned by the incorporation of EVs with cationic, anionic, and zwitterionic lipid chains.

To evaluate the difference of lipid:EV mixture before and after the sonication-extrusion processes, zeta potentials of the vesicles were measured, as shown in FIG. 2I. The data showed the sonication-extrusion processes significantly alters the zeta potential of the samples in comparison to the samples of physical mixture (**p<0.01 and ***p<0.001 for POPC-EV 4:1, 1:1 and 9:1 group, respectively). The zeta potentials of the eEVs at varying extruded ratio were also statistically different (**** p<0.0001) compared to the native exosomal membrane (FIG. 2I). The zeta potentials of EV and eEVs measured in PBS and water were further characterized, as shown in FIG. 2G. It is known that phospholipids, such as cholesterol, phosphoglycerides, ceramides and saturated fatty acids, are rich within EV membranes (79). The presence of saturated phospholipids and the anionic surface charge help contributed to the high stability of EVs. Therefore, relatively negative zeta potentials of EVs were obtained with −22.9±0.6 and −17.3±1.4 mV in water and PBS, respectively. Compared to the zeta potentials of samples measured in pure water, zeta potentials in PBS were closer to neutral (0 mV). These results can be attributed to the charge shielding effects by salt ions present. These data are similar to the zeta potential of EVs generated from the neuroblastoma cell lines reported previously (80), which ranged from −14.8±1.6 to −12.0±0.2 mV in a PBS solution. On the other hand, the zeta potentials of eEVs were found to correspond to the charge of extruded lipids. DOTAP-EV, POPC-EV and DPPC-EV, and POPG-EV showed positive, neutral, and negative charge, respectively (FIG. 2G), indicating the zeta potentials of eEVs can be tuned by hybridizing EVs with different charge types of synthetic lipids. Taken together, this data demonstrate the sonication-extrusion processes significantly alters the zeta potential of eEVs, suggesting that there were substantial alterations of the lipid content within the eEVs.

### EXAMPLE 3

### Quantifying Native EV Fraction Within eEVs for Validating Membrane Incorporation

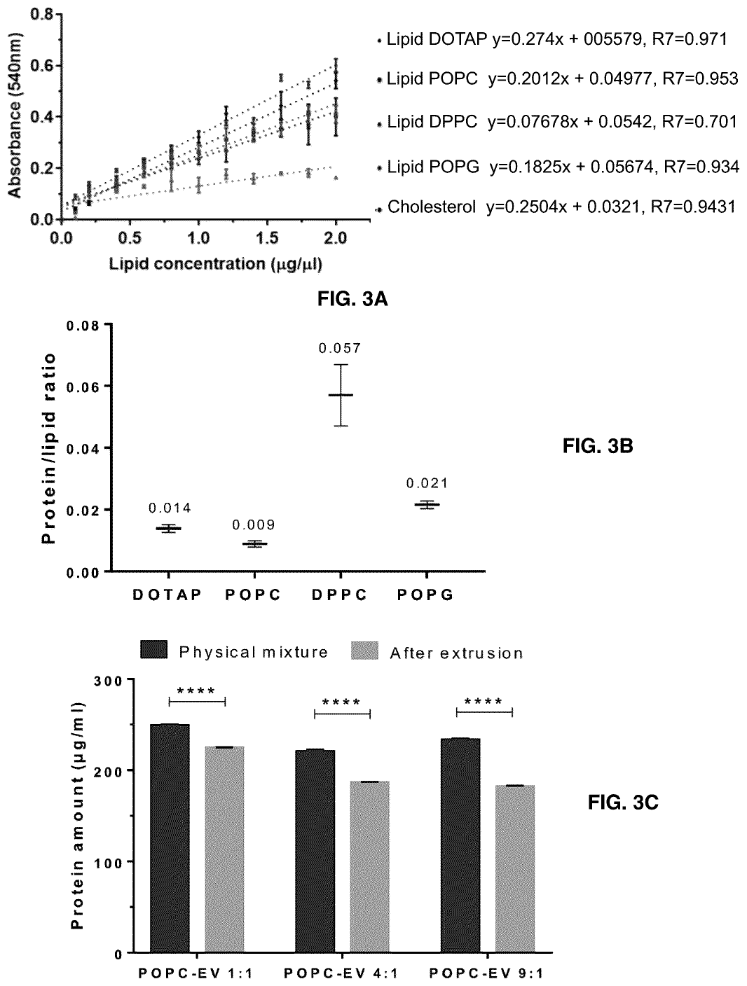

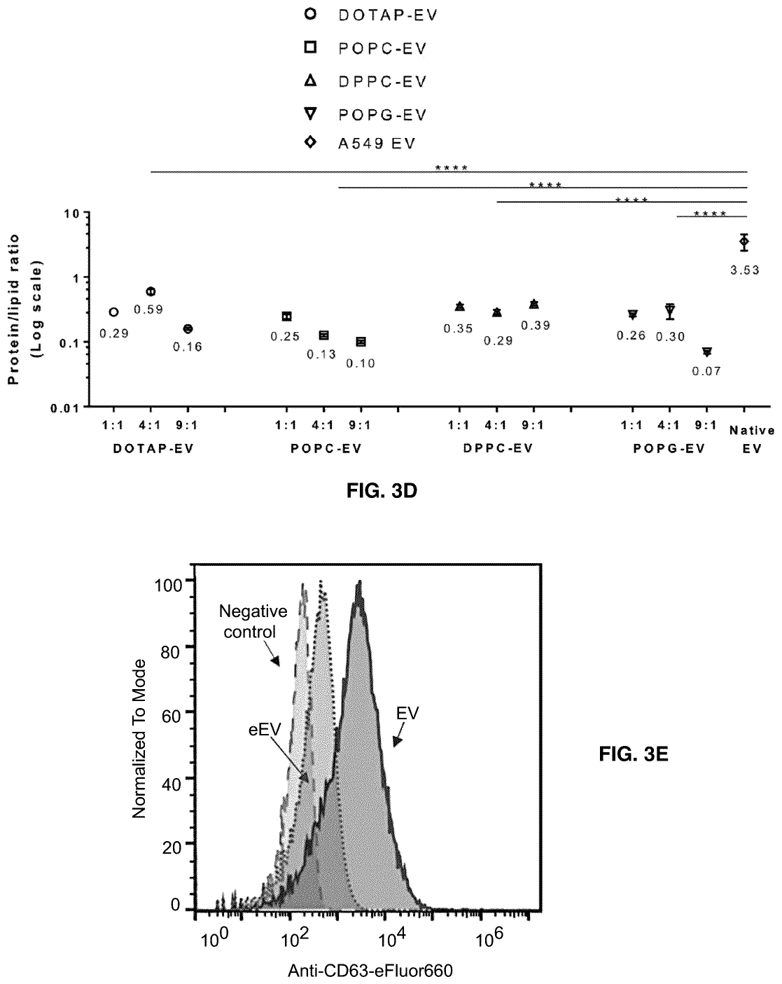

The lipid (SPV) and protein (Micro BCA) assays that are widely used in the EV field were chosen to determine membrane incorporation within the vesicles. Before determining the protein to lipid ratios in EVs/eEVs, preliminary tests of individual lipid species were measured as references, which showed good linearity of absorbance in the range of 0-2 μg/μl lipid concentration using SPV assay (FIG. 3A). In parallel to the lipid quantification, pure lipid solutions were also tested for their assay interference with the protein determination (FIG. 3B). No substantial interference on the protein Micro BCA assay by lipids was observed, as the data did not result in strong colorimetric reactions. The protein quantities between the physical mixtures of EV: lipid and the eEVs were evaluated following sonication-extrusion processes. FIG. 3C evidences a statistically significant change (****p<0.0001) of protein quantities in the lipid-EV mixture before (physical mixture in the absence of sonication-extrusion processes) and after sonication-extrusion processes (lipid-doped eEVs). To further determine the fusion efficiency of exosomal membrane in eEVs, the protein to lipid ratio was quantified, which has been proven to be a good quality control parameter of EVs previously (46). FIG. 3D presents the calculated protein to lipid ratios of EVs/eEVs. The protein to lipid ratios of eEVs (0.069±0.004 to 0.594±0.055) significantly (****p<0.0001) dropped compared to the protein to lipid ratio of EVs (3.529±1.015). These results demonstrate the exogenous synthetic lipids were doped within EVs. With the addition of lipids to EVs, the overall percentage of lipids within the membrane increases, results in the dropping of the protein to lipid ratios within eEVs. In comparison to the pure lipid solutions (FIG. 3B), these results showed a 5-fold (DPPC-EV 4:1/DPPC lipid=0.29/0.057) to 42-fold (DOTAP-EV 4:1/DOTAP lipid=0.59/0.014) of protein to lipid ratio change within eEVs upon the membrane extrusion procedure. Note that the protein to lipid ratio only slightly changed at varying extruded ratio, as no statistical difference was observed (FIG. 3D). Similar trend of the protein to lipid ratio within eEVs was obtained while using the EVs from different cell sources (3T3 EV and A549 EV) (FIG. 3C), indicating this technique for membrane incorporation can be applied to EVs derived from different parental cells.

To further confirm the membrane fusion of exogenous synthetic lipids within the EVs, as opposed to synthetic lipids and EVs being physically mixed, flow cytometry was used to detect the fluorescence of each entity. The histogram plot of anti-CD63 fluorescence is shown in FIG. 3E. Normalized geometric means of fluorescence confirmed the percentage of membrane incorporation in POPC-EV (1:1, 4:1, 9:1) samples as a fraction ratio of 0.16 to 0.21 (FIG. 3F), meaning the eEVs having 16-21% native EV membranes within them. Similarly, no statistical difference was observed at varying extruded ratio for eEVs. The results of the flow cytometry-based assays and the membrane protein/lipid assays corroborate each other, in that both illustrate the instant sonication-extrusion process fused a fraction of each of the synthetic lipids within the EVs. The histogram plot of anti-CD63 fluorescence in flow cytometry demonstrated individual eEVs present higher CD63+ levels compared to the control group of liposome-POPC as background fluorescence and lower levels compared to native/naïve EVs, suggesting the successful fusion of exosomes with lipids. These data demonstrate that the instant method of generating EVs can increase the yield of particles post-EV isolation while retaining native protein (i.e. CD63) within each entity.

It is interesting that statistical differences of eEVs at varying extruded ratios in both protein/lipid assays and flow cytometry experiments were not observed, indicating it was likely at a point of saturation, in terms of the amount of synthetic lipid that could be incorporated into the membrane. These results are different from the lipid-hybridized EVs triggered by PEG-induced fusion reported from literature (77), who showed that 9% to 56% of lipids could be incorporated by varying EV: lipid ratios. This discrepancy between the two methods could likely be attributed to the difference of lamellarity and localization of lipids within the EVs. While 30-40% of multi-lamellar vesicles tend to form using PEG-induced lipid:EV fusion, additional studies for lipid-doped eEVs using sonication-extrusion processes would need to be conducted to suggest a percentage.

### EXAMPLE 4

### EV and eEV siRNA Loading

The capability of EVs for efficient drug loading without drastic physicochemical modification of the native vesicles is one of the major and practical obstacles in the clinic. After generation of lipid-fused eEVs, encapsulation of exogenous siRNA into the vesicles was attempted. As a first step in evaluating the efficacy, the changes of physicochemical properties were assessed following electroporation. The eEVs post-siRNA loading via electroporation had intact and round shaped morphology. Occasionally, the vesicles appeared to be fused or aggregated after electroporation. No obvious alternations on the vesicle size of the samples after electroporation were observed by NTA measurement at the concentration of ˜108 particles per ml. However, there was a statistically significant change (*p<0.05) on the z-average diameters of the vesicles post-electroporation, as measured by DLS at concentration of ˜10¹¹ particles per ml. The zeta potentials of eEVs after electroporation significantly decreased (**p<0.01) from −4.9±3.5 mV to −28.0±3.5 mV. Possible reasons for the differences between the results from the two instruments could be that DLS is biased towards large particles or aggregation occurs at higher concentration of the samples required for DLS measurement (˜10¹¹ particles/ml) compared to the sample concentrations required for NTA measurements (˜10⁸ particles/ml).

Further the actual siRNA amount loaded into the vesicles was assessed. Prior to siRNA purification and quantification procedures, free unbound siRNAs in the sample mixture were removed by centrifugation and wash. By repeating these steps, the data showed only 1.2±0.2% of free siRNA retained in the pellet after centrifugation, validating the efficacy of removing free unbound siRNA. To evaluate siRNA loading efficiency via electroporation, it was first demonstrated that the buffer used for electroporation (hypotonic buffer vs isotonic Opti-MEM) does not substantially affect siRNA loading efficiency with no statistical difference), but does affect the retention of siRNA within the vesicles. The siRNA was confined in the sample well with the electroporation condition using Opti-MEM media, resulting in a lower siRNA migration amounts than the samples using hypotonic electroporation buffer. Using Opti-MEM media, siRNA was retained and bound within the vesicles. Based on these findings, Opti-MEM was used for all further evaluations.

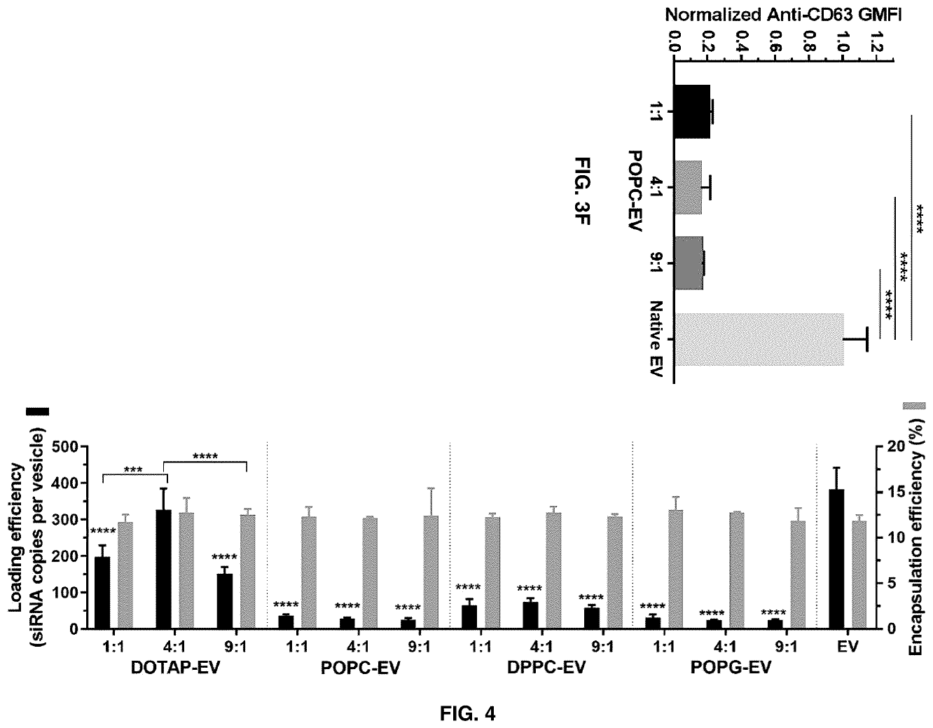

Subsequently, the loading efficiency (FIG. 4, left y-axis) and encapsulation efficiency (FIG. 4, right y-axis) of each lipid-doped eEV system was quantified. It was demonstrated that exogenous siRNA can be loaded into eEVs and the loading efficiency is dependent on the hybridized lipid types. The loading efficiency results showed that 23-327 copies of siRNA per vesicle were obtained. Particularly, DOTAP-EVs showed highest loading efficiency, which was at least 8-fold higher than other lipid-extruded eEV systems. Moreover, the loading efficiency of cationic lipid-doped eEVs is comparable to the loading efficiency of unmodified, native EVs (FIG. 4; no statistical difference). Taken together, zwitterionic eEVs (POPC-EV and DPPC-EV) and anionic eEVs (POPG-EV) exhibit generally comparable loading profiles (no significant difference between each other).

Consistent with the previous findings (48) similar electroporation efficiencies were obtained (˜15-20% of siRNA could be encapsulated in EVs/eEVs by electroporation). Overall, electroporation seems to be a robust method for cargo loading within the eEVs, albeit aggregation issues might be a concern for cell studies.

### EXAMPLE 5

### Mitigation of eEV Aggregation and Its Effects on Cytotoxicity

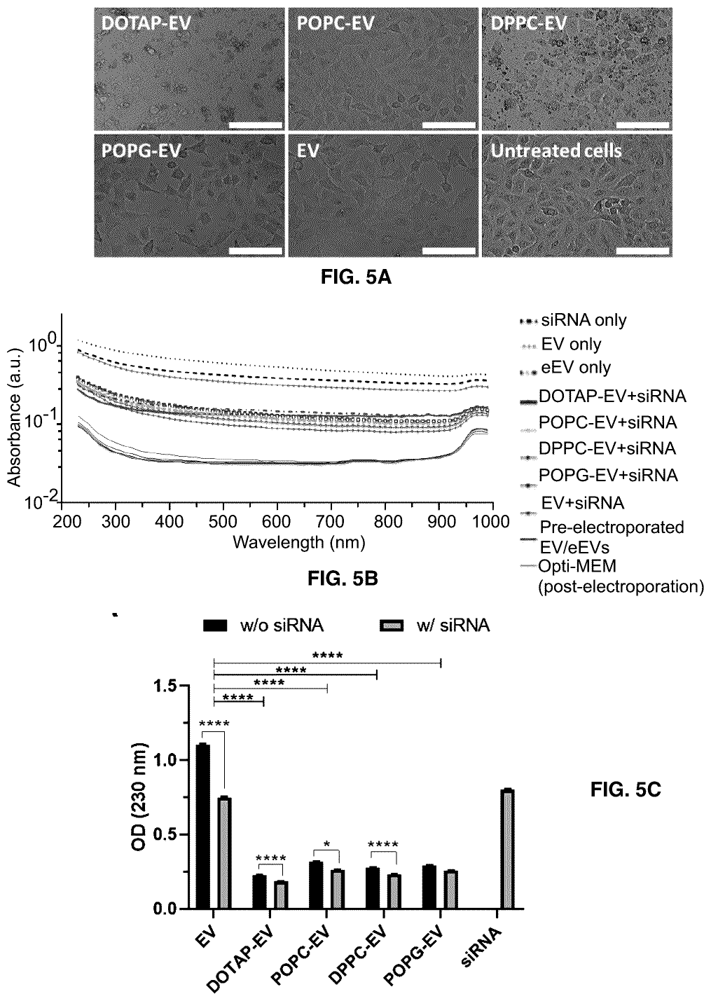

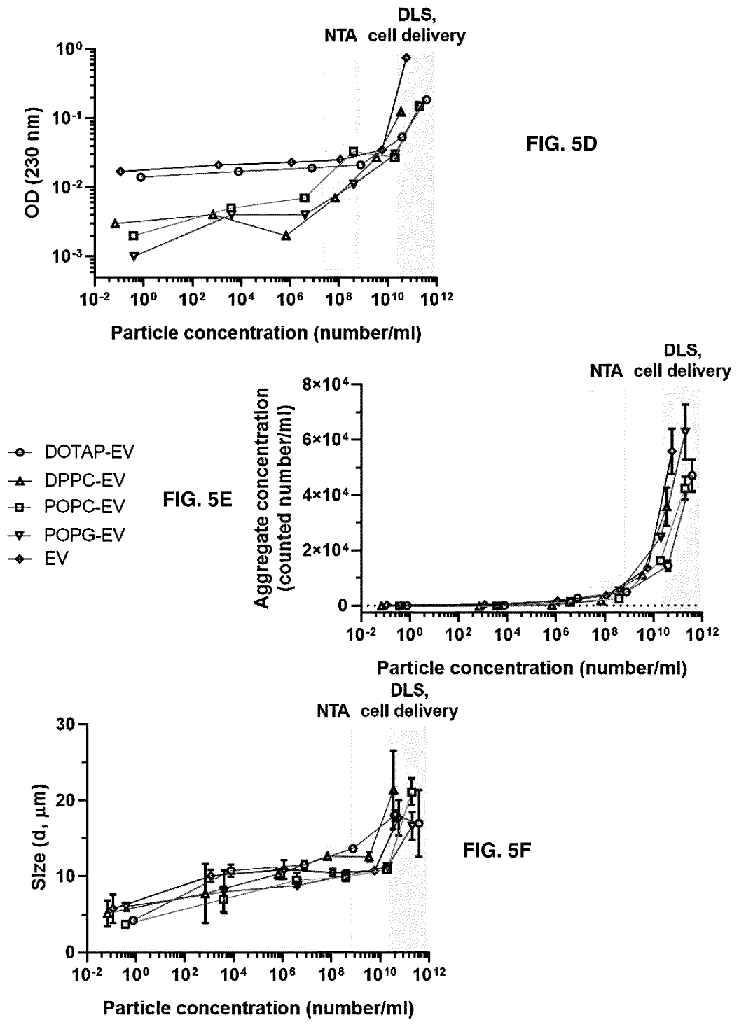

A more stable (in comparison to what was initially commercially procured), and higher GFP-expressing tumor cell model (A549) for in vitro cell studies was established. This GFP model enables one to easily visualize and quantify the delivery of anti-GFP siRNA, and the resulting knockdown of the GFP using fluorescence microscopy, fluorescence plate readers, and flow cytometry. The cell viability (MTS) assay was first conducted to evaluate if there is any cytotoxicity issue with siRNA loaded EV/eEV (10¹⁰-10¹¹ vesicles/ml) post-electroporation. Unexpectedly, independent of the lipid-doped eEV used, the viability was lower than 70%. Although in Example 4 DOTAP-EVs were associated with higher siRNA loading efficiency, here it resulted in the highest toxicity, ranging from 18.2±3.8% to 56.5±3.9% at varying lipid:EV ratio. The cytotoxicity of DOTAP-EV could potentially be attributed to the charge of lipids, which has been shown to be toxic to the cells at high concentrations, although it is commonly used as a transfection agent. In addition to the charge effects from the lipids, it was hypothesized these cytotoxic results likely could be attributed to particle aggregation and sedimentation of the eEV-siRNA delivery system, as was seen in the brightfield images (FIG. 5A), note the precipitation of eEV within the bright field image of the engineered DOTAP-EV and DPPC-EV formulation. It is currently believed that the electric pulses from electroporation may cause undesirable metal ions to release from the electrodes and subsequently affect the lipid oxidation and the solubility of a variety of biomolecules. To determine whether the toxicity was due to the electroporation process or not, the cytotoxicity of eEVs with siRNA mixture in the absence of electroporation was quantified. The viability remained high (˜80-125%) and was relatively independent of particle concentration. There were no obvious signs of toxicity observed in A549 cells while increasing the eEV concentrations from (0.13-1.08)×10¹¹ and (0.95-15.72)×10¹¹ particle/ml for the engineered DOTAP-EV and POPG-EV formulations, respectively. However, there was a significant reduction of cell viability for both eEV-siRNA mixture assessed post-electroporation: DOTAP-EV (post-electroporation) decreased 7-fold to only 18% viability (***p<0.001) and POPG-EV (post-electroporation) decreased 2-fold to 51% viability (**p<0.01). These results validated that the electroporation processes may cause undesirable consequences, such as aggregation, resulting in high cytotoxicity.

### Aggregation Evaluation by Spectroscopic Assays and Microscopy Image Analysis

To quantitatively determine the degree of aggregation post-electroporation, spectrometry and microscopy techniques were used. FIG. 5B shows the UV-Visible spectra of siRNA, EV, and various lipid-doped eEVs. In all cases, the samples were electroporated either with (“+siRNA”) or without siRNA (“vesicle only”). The Opti-MEM and pre-electroporated eEVs conditions were measured as baselines and their spectra appear to overlap substantially. Because there was not a signature absorbance peak in the spectra, the maximum absorbance at 230 nm was used to quantify the aggregation as a turbidity measurement (57, 81), as shown in FIG. 5C. The results from both experiments showed the electroporation-induced aggregation occurred in not only membrane vesicles but also in naked nucleic acids (siRNA). These findings were in line with previous observations (82,83). Notably, the quantitative results of turbidity (OD: 230 nm) (FIG. 5C) showed a decrease of the tendency of aggregation when the electroporation is carried out in the presence of both siRNA and vesicles, compared to electroporating vesicles/siRNA alone. Moreover, the data suggest that in most cases, the degree of aggregate formation decreased for all lipid-doped eEVs, compared to native EVs post-electroporation. A larger library of lipids need to be assessed in order to elucidate the entire parameters affecting the aggregation.

The aggregation of siRNA loaded EVs/eEVs, in terms of the turbidity (OD: 230 nm), number, and the size of aggregates, was validated. The results vary in a concentration-dependent manner for all EV/eEV formulations. The data demonstrated the degree of aggregation (FIGS. 5D-5F, y-axis are OD: 230 nm, aggregate concentration, and size, respectively) significantly increased 9- to 17-fold at the concentrations used for DLS and cell studies (>10¹⁰ particles/ml), in comparison to the sample concentration used for NTA measurement (10⁸ particles/ml). These findings point out the electroporation-caused aggregation may be far more significant than previously believed. Given that NTA is commonly used for particle analysis in the scientific community at a concentration of approximately hundred-fold lower than the dose applied for cell studies, the aggregation effects are likely underestimated. This provides evidence that strong aggregation of samples occurs after electroporation, which may result in far more severe impacts for in vitro and in vivo studies than NTA appears to be suggesting.

### Optimization of the Electroporation Processes for Efficient Cargo Loading and Alleviation of the Electroporation-Caused Aggregation and Cytotoxicity

Because substantial aggregation of EV/eEV and siRNA was formed following electroporation, mitigating the toxicity by tuning the electroporation conditions, i.e., the parameter specific to the electroporation buffer, was examined.

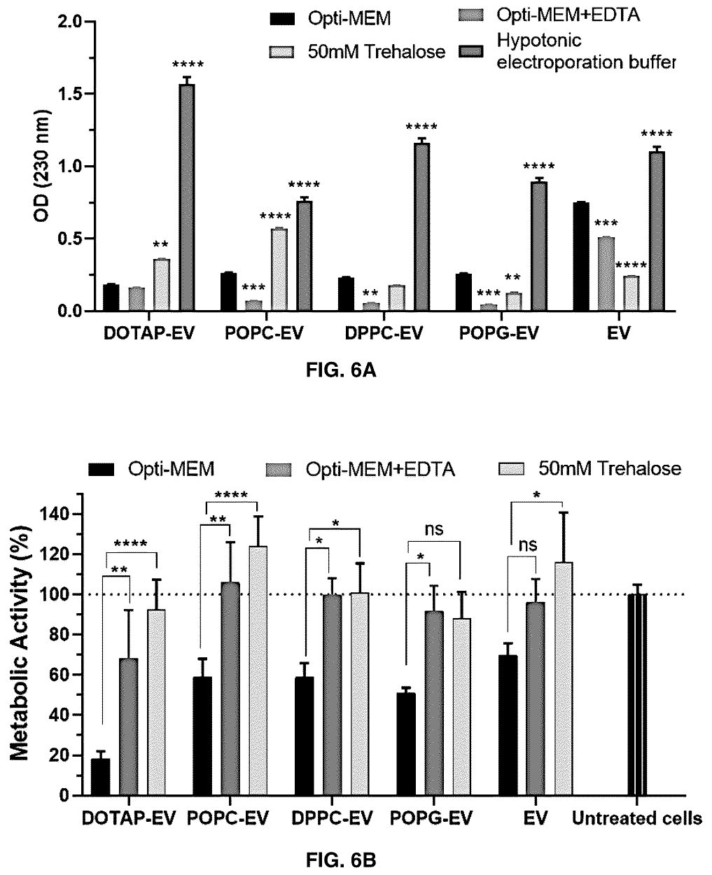

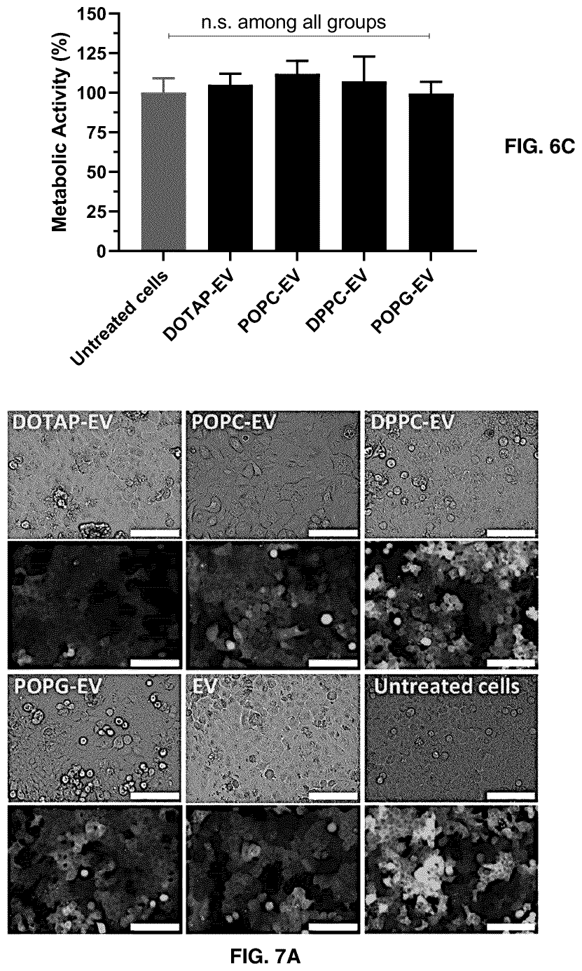

The aggregation effects of various electroporation media (Opti-MEM+EDTA, 50 mM trehalose, and hypotonic electroporation buffer) after electroporation were examined. It was demonstrated the aggregation can be markedly decreased by adding EDTA to the electroporation buffer (FIG. 6A), as suggested in previous literature (82,83). However, the microscope imaging results confirmed the submicron aggregates were not removed and yet still existed in substantial amounts by microscopy image analysis. More strikingly, the effects of 50 mM trehalose on the aggregation level appeared to be different for different lipid-type eEVs. While the aggregation level of DPPC-EV, POPG-EV and native EV tended to decrease in 50 mM trehalose following electroporation, unexpectedly, the aggregation of DOTAP-EV and POPC-EV were likely to increase (FIG. 6A).

These results are opposite to the hypothesis from Hood et al. who reported that a 50 mM trehalose solution may minimize aggregate formation following electroporation (61), suggesting different concentration of trehalose solution may be needed for different lipid-doped eEV system to optimize the aggregation effects. Moreover, despite hypotonic electroporation buffers being commonly used in several studies for electroporation, it was demonstrated here that the aggregation of eEVs in hypotonic electroporation buffers following electroporation was at least 1.5-fold higher than the eEVs in Opti-MEM (FIG. 6A). This discrepancy in the results compared to previous literature may be attributed to the differences of employed assays for aggregation evaluation. While most studies used NTA measurement as a mean to determine the size and numbers of aggregates, the sub-visible aggregates may be underestimated, as discussed in the previous section.

Regarding metabolic activity of EV/eEV while varying the electroporation buffers (FIG. 6B), the 50 mM trehalose formulation was affiliated with the highest viability, except for POPG-EV (no statistical difference). In all cases, the formulations using Opti-MEM were significantly more toxic than the other buffers, which was 2-fold more toxic than using Opti-MEM+EDTA and 50 mM trehalose. Taken together, it is interesting to note that these results demonstrated the degree of aggregation of electroporated mixture may not be a direct linear relationship corresponding to cytotoxicity. The trend of cell viability with the variation of aggregation was demonstrated by combining the data from various media. The correlation of aggregation for eEV appeared to be biphasic in response to cell viability, while the most toxic regime showed in the range of 0.2-0.4 measured absorbance (OD: 230 nm). Importantly, it should be noted that elucidating whether aggregation and cytotoxicity are correlative, or causative is challenging, as aggregation is a secondary variable dependent on other factors. All other variables cannot be held constant while varying aggregation to assess its effects on cytotoxicity alone. It is important to note that the cytotoxicity profiles of eEVs on non-cancerous cells also were examined. A concentration of 10¹⁰-10¹¹ vesicles/ml were used as a single dose in Opti-MEM containing 1 ng/μl siRNA. 5 mM EDTA was added to the samples after electroporation. No toxicity was observed on healthy CCL-210 cells using the optimized electroporation buffer (FIG. 6C). Given that the promise of electroporation continues to be hampered by a lack of appropriate and optimized conditions, it is contemplated that examining these parameters may be helpful for future consideration of EV-based gene therapies and enhance the potential clinical translation of EVs.

### Quantification of RNAi Knockdown and eEV Targetability

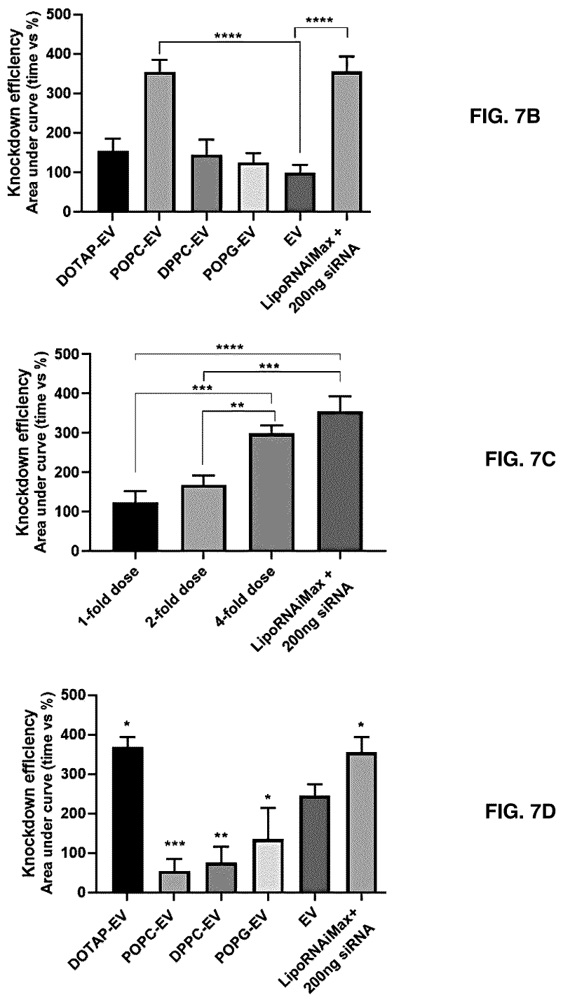

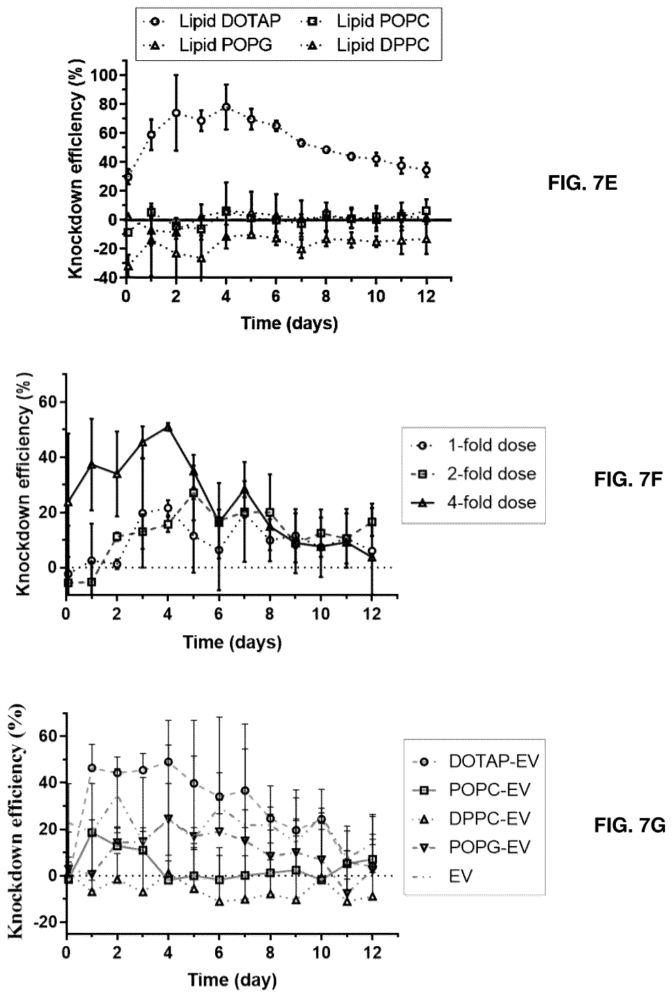

The siRNA silencing effects of EVs and eEVs electroporated in both Opti-MEM media with EDTA addition and 50 mM trehalose with the formulations which were affiliated with a cell viability greater than 80% (FIG. 6B) were assessed. FIG. 7A qualitatively demonstrated the decrease in the total GFP expression in the cells. To quantify the GFP expression in cells, fluorescence intensity was monitored over time using a plate reader assay. FIGS. 7B-7D quantified the overall knockdown efficiency by analyzing the area under the curve for knockdown over time. Using 100 μl Opti-MEM, 10¹¹ particles/ml, and 1 ng/μl siRNA in Opti-MEM, a 10-46% of knockdown efficiency was able to achieve by the eEV formulations, whereas the native EV-delivered siRNA failed to knockdown the GFP expression in an effective manner. An early and rapid decrease of knockdown efficiency was observed in native EV treated group at day 2, while the knockdown efficiency dropped after day 4 in eEVs. Overall, zwitterionic POPC-EV showed a highest knockdown with 41.1±9.8% at 4 days post-transfection, which was comparable to commercial Lipofectamine RNAiMax of 43.3±0.3%. A summary of the overall knockdown efficiency in FIG. 7B showed the knockdown efficiency of POPC-EV over time was statistically greater than the knockdown efficiency of EV (****p<0.0001). Next, the experiments were repeated using anionic POPG-EV to deliver the siRNAs to the cells with different dosage. Despite only 21% of knockdown was achieved by POPG-EV with initial dosage, FIG. 7F showed that high dose of eEVs can more effectively down regulate the expression of GFP. A 2.4-fold of increase in inhibition of GFP expression was observed for 4-fold amount of dose of POPG-EV compared to initial dose. No statistical difference was observed between the area under curve of POPG-EV with the amounts of 4-fold dose and commercial Lipofectamine RNAiMax (FIG. 7C). The knockdown results among different lipid-doped eEVs implied the minimum effective dosage required varied with the properties of hybridized lipids. Further evaluation on the dosage effect would be worthwhile in future studies.

On the other hand, for the knockdown results of EVs and eEVs electroporated in 50 mM trehalose (FIG. 7G), the extent of knockdown of EV increased to 22-34% knockdown compared to the knockdown results (<10%) electroporated in Opti-MEM. These results could potentially be attributed to the decrease of aggregation for EV in trehalose. However, like the previous aggregation data, the effects of the electroporation buffer on knockdown also differed between different lipid-doped eEVs. A significant increase of knockdown efficiency to 49.0%±7.3% was observed in the cells treated with cationic DOTAP-EV in 50 mM trehalose at 4 days post-transfection, while POPC-EV failed to reach 10% knockdown of the GFP expression (FIG. 7G). The overall knockdown efficiency of eEVs in Trehalose (FIG. 7D) shows only the knockdown efficiency of DOTAP-EV over time was comparable to the knockdown efficiency of commercial Lipofectamine RNAiMax (no statistical difference) and greater than the knockdown efficiency of EV (*p<0.05) in this case.

While discussing the functionality and efficacy of eEVs using different electroporated media, it should be noted that the findings of electroporation-induced aggregation pointed out a complication regarding the determination of knockdown efficiency; aggregated and precipitated particles may be uptake differently by the cells and may not be easily removed by washing. Therefore, it can be mistakenly considered as being transfected into the cells (82). Further, it seems that the scientific community has not conducted experiments to determine whether such aggregates inhibit or enhance the EV-mediated siRNA delivery into the cells (83). Aggregation is known, however, in previous studies to cause enhanced uptake in at least certain cases. Interestingly, by assessing the knockdown effects of lipid-siRNA complexes, which were the same lipids used for doping purposes, it was found RNAi only occurred when lipids POPC, POPG and DPPC were fused into EV vesicle membrane. As shown in FIG. 7E, siRNA complexes with lipid POPC, POPG and DPPC were not able to down-regulate GFP expression. These results suggest the lipoplexes which may form would not have contributed to the knockdown observed for POPC, POPG, and DPPC lipid-doped eEVs.

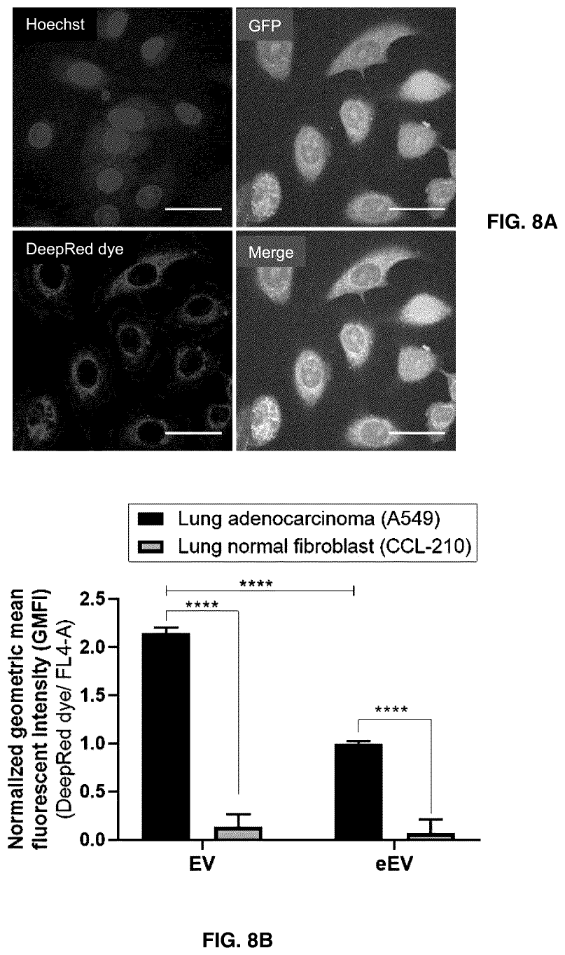

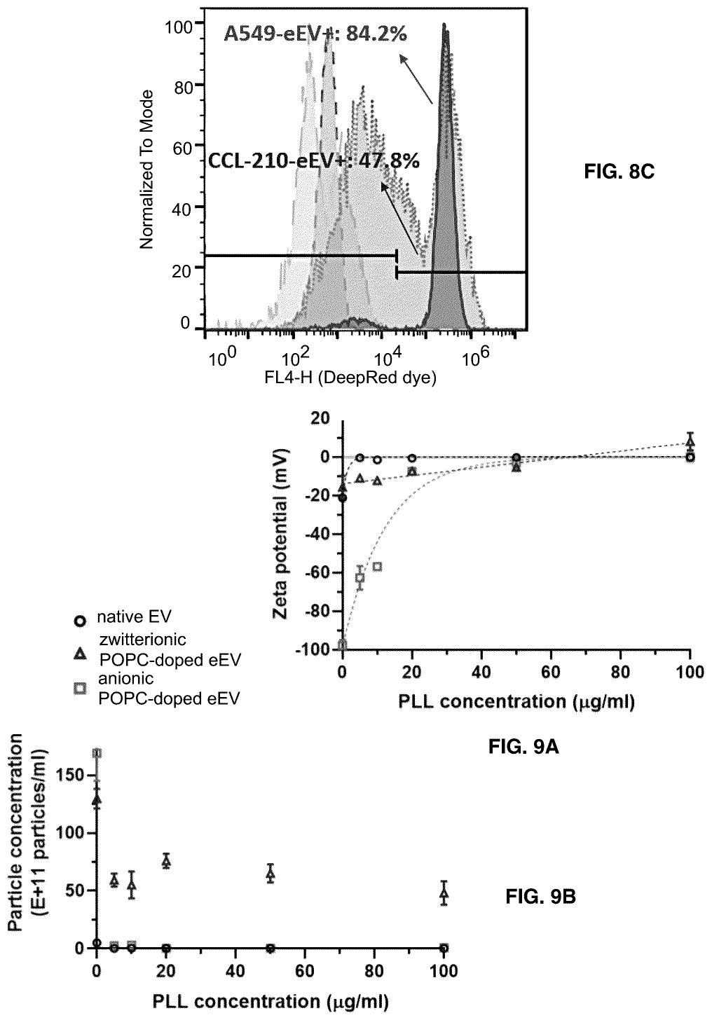

To further investigate if lipid-doped eEVs can direct targeting to different cell lines, the cellular uptake of native EVs and eEVs in lung adenocarcinoma (A549) and lung normal fibroblast (CCL-210) were quantified. Prior to the uptake studies, Deep Red membrane dye was used to stain the exosomal membrane of EVs/eEVs, followed by incubation with the cells. The confocal images of cellular uptake (FIG. 8A) revealed the internalization and localization of vesicles (lower left) in the cytoplasmic area. To further quantify the uptake efficiency between cell lines, flow cytometry assay was conducted and demonstrated in FIG. 8B. Significant differences in EV uptake between the two cell lines were observed, with the most efficient cell line, lung adenocarcinoma (A549), demonstrating a 15.8-fold higher uptake amount compared to the normal lung fibroblast (CCL-210). Remarkably, A549 cells also showed an increased propensity to take up eEVs that a 14.2-fold higher eEV uptake efficiency by A549 cells than that by CCL-210 cells was observed. Moreover, A549 cells exhibited a higher percentage of uptake population than CCL-210 cells. Approximately 84.2% of A549 cells were uptake with eEVs, whereas only 47.8% of CCL-210 cells were uptake with eEVs (FIG. 8C).

Taken together, a universal increase in uptake efficiency for the lung cancer cells versus normal lung fibroblast was observed. These results supported the conclusion that the lipid-doped eEVs retained the targetability of EVs, despite the degree of selectivity on the cell lines could decline by the decrease of the EV portion in the engineered vesicle membrane. It is contemplated that how the engineered lipid-doped eEVs entered the cells and how knockdown is achieved should be investigated.

The cellular internalization of eEVs using zwitterionic POPC-EV was tested because of the high knockdown efficiency (FIG. 7B) and because little to no gene delivery effect from POPC lipid-siRNA complexes was in the previous evaluation (FIG. 7E) that the cellular internalization would less likely be affected by any free lipid components. It is contemplated that more research should be done in terms of the uptake mechanisms (i.e. clathrin- and caveolae-mediated endocytosis, macropinocytosis) to elucidate the critical factors determining EVs/eEVs uptake and targeting at molecular structure levels.

### EXAMPLE 6

### Fabrication and Optimization of Multi-Layered Engineered Extracellular Vesicles (LbL-eEVs)

The multi-layered engineered extracellular vesicle (LbL-eEV) platform consists of two main components: (1) engineered lipid-hybridized extracellular vesicles (eEVs) as the carrier of siRNA and (2) tri-layered shell assembly of polyelectrolytes as the carrier of chemotherapeutics (DOX). EVs were collected from A549 cells and hybridized with either zwitterionic or anionic phospholipids to create eEVs. The lipid-hybridized eEVs were generated via sonication and extrusion according to the protocol in prior work (63). Multi-layered polyelectrolyte shells were then assembled on the surface of the eEVs to produce LbL-eEVs.

PLL, PAA and PBAE were selected as cationic and anionic counterparts to form a tri-layered shell sequentially surrounding the core eEVs. The polycation PLL was applied as the first layer because the extracellular vesicle surface was weakly anionic. PAA was applied next and enabled incorporation of the small molecule drug DOX, which contains an amino group with a pK of 8.6 and, thus, will bind strongly to the carboxylate groups on PAA. PBAE, which is a superior biocompatible and bio-reducible polymer compared to non-degradable polymer PEI (41), was chosen to facilitate cytoplasmic targeting of siRNA release.

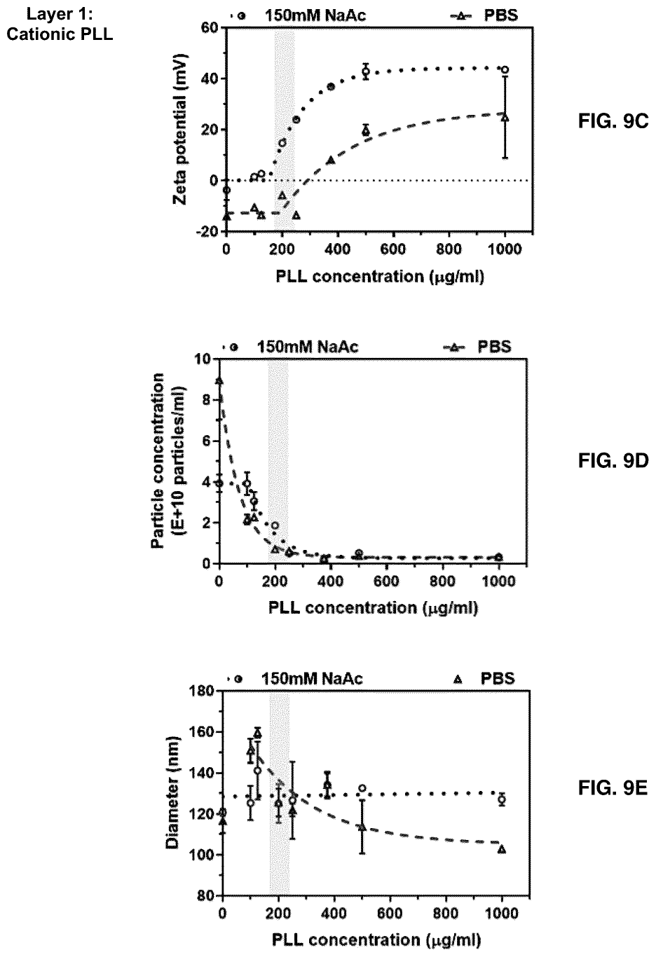

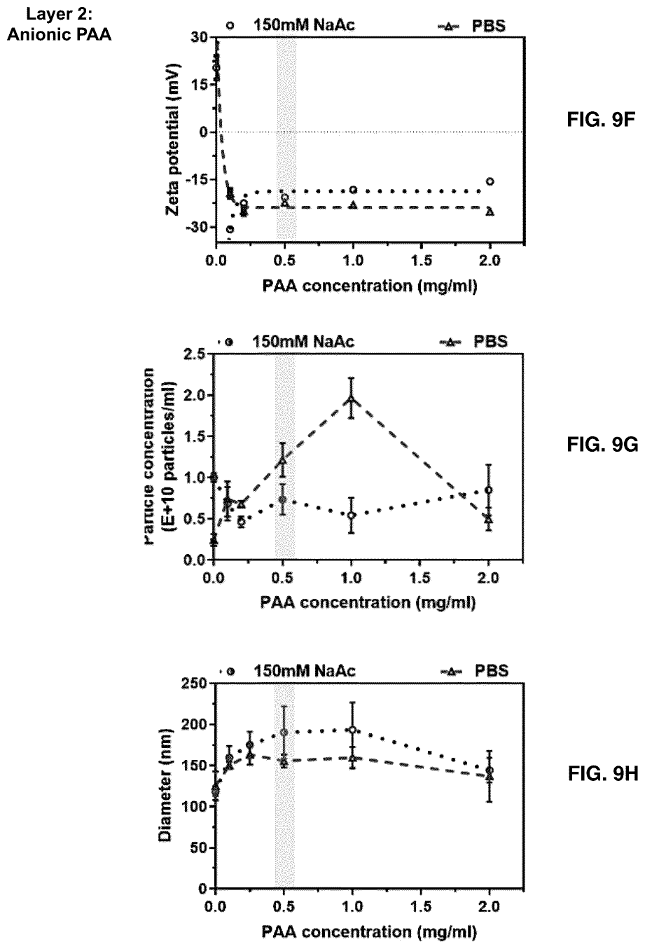

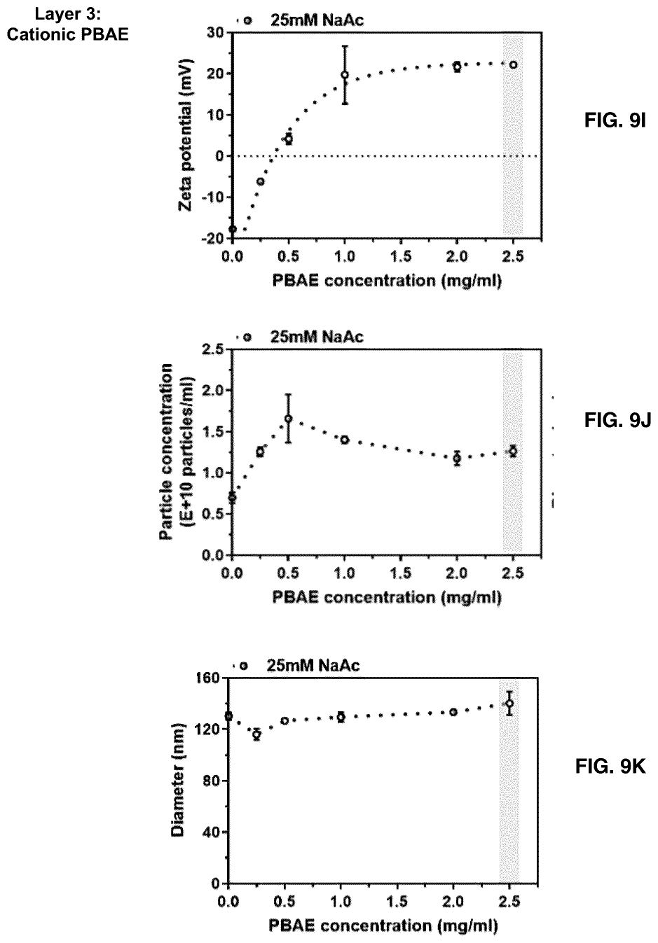

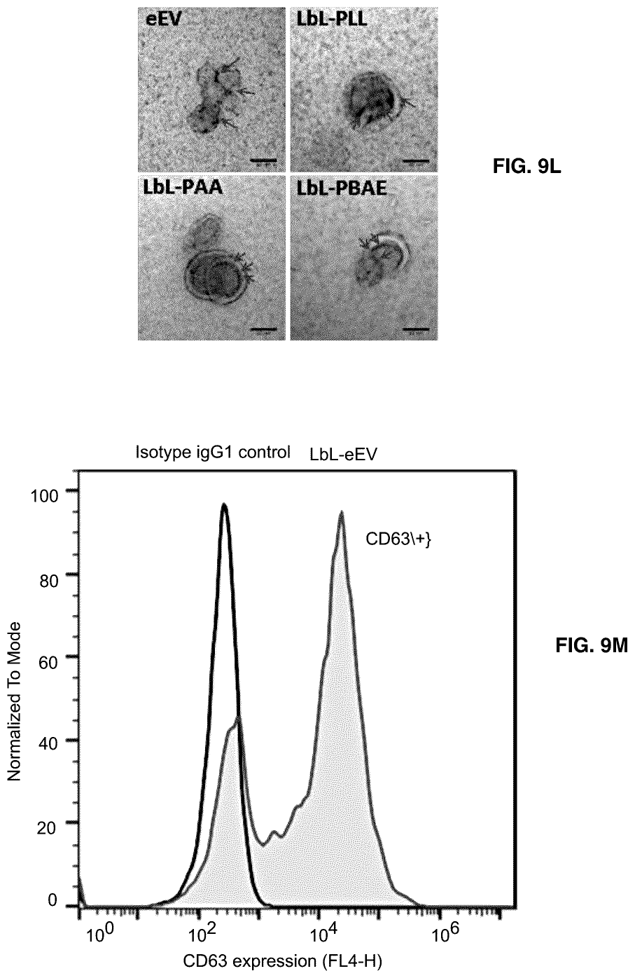

To produce stable LbL-eEVs, the LbL deposition procedures on native and lipid-hybridized eEVs, specifically zwitterionic POPC-doped eEVs and anionic POPG-doped eEVs were studied. FIGS. 9A-9B show the change in zeta potential and particle concentration of the eEV mixtures following PLL deposition. A successful reversal of charge was only observed with the zwitterionic POPC-doped eEVs. The increase in zeta potential leveled off at 5 μg/ml and 50 μg/ml PLL concentration for native EVs and anionic POPG-doped eEVs, respectively, and charge reversal was not achieved (FIG. 9A). In addition, the particle concentration after PLL coating (FIG. 9B) was found to decrease 22-fold and 172-fold for native EVs and anionic POPG-doped eEVs, respectively, whereas zwitterionic POPC-doped eEVs did not exhibit such drastic decreases in particle number following cationic PLL deposition. Therefore, zwitterionic POPC-doped eEVs were used for subsequent experiments.

Next, optimizing the polyelectrolyte assembly on core eEV materials, solutions for polyelectrolyte deposition and the effects of polyelectrolyte concentration were investigated. The zeta potential, particle concentration and diameter were investigated after each coating step, as shown in FIGS. 9C-9K. First, the influence of phosphate buffer saline (PBS, pH 7.4) and 150 mM sodium acetate (NaAc, pH 5) on polyelectrolyte deposition were compared. By simply decreasing the pH during polyelectrolyte incubation, a lower threshold for inversion of surface charge was obtained (FIG. 9C). Therefore, 150 mM NaAc was selected for the first and second layer of polyelectrolyte coating. It is worth noting that 25 mM NaAc was used instead of 150 mM NaAc in the final layer (PBAE) to reduce acidity for later use in cellular studies, according to the prior studies (42). Second, the influence of polyelectrolyte concentration on polymer-EV complexes was examined. In all cases, the zeta potentials (FIGS. 9C, 9H and 9K) of coated eEV substances changed in an exponential growth/decay kinetics, followed by a plateau, indicating a saturation of the charge density. Notably, while a layer of polyelectrolyte is built up on the surface, the particle concentration (FIG. 9D, 9G and 1J) of polymer-EV complexes decreased, as a result of charge overcompensation. However, a statistically significant effect on particle diameter (FIGS. 9G, 9J and 9K) at varying polyelectrolyte concentrations was not observed.

The results demonstrated a balance between charge reversal and particle colloidal stability in terms of the optimal concentration of polyelectrolyte is crucial for the success of LbL deposition. These results are consistent with previous studies (85-86). Based on the point of charge inversion and particle concentration, concentrations of 0.2 mg/ml, 0.5 mg/ml and 2.5 mg/ml were selected for PLL, PAA and PBAE, respectively, for subsequent experiments. The structures of LbL-eEVs are shown in the transmission electron microscopy (TEM) images (FIG. 9L). The exposure of tetraspanin surface proteins after application of the LbL coating to the eEVs was confirmed by performing anti-CD63 labeling and on-bead flow cytometry (FIG. 9M).

### EXAMPLE 7

### SiRNA Loading in the eEV Core and Small Molecule Drug Loading in Polyelectrolyte Shells

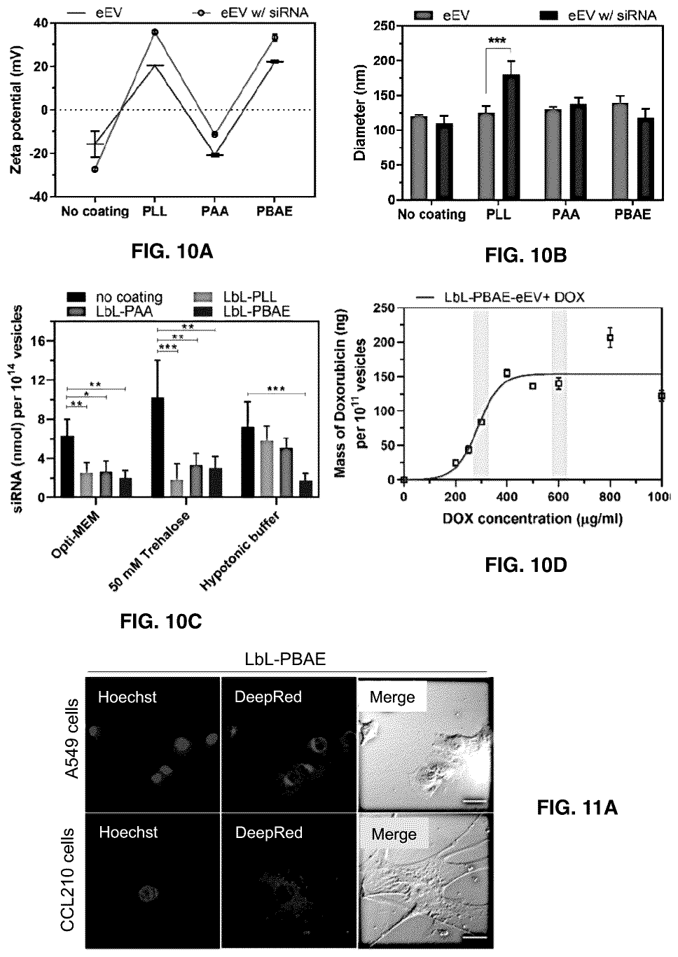

Benchtop electroporation systems have been widely used to encapsulate small nucleic acids such as siRNAs into EVs (88). In order to develop a co-delivery platform for siRNA and small molecule drugs in this study, siRNA was loaded into eEVs via electroporation prior to polyelectrolyte shell assembly. The characterization results from stepwise coating of eEVs with and without siRNA are summarized in FIGS. 10A-10D. FIG. 2A shows the change in surface charge as each polyelectrolyte layer is deposited. While siRNA-loaded eEVs exhibited a greater negative charge of −27.3±0.8 mV compared to eEVs without siRNA (−15.7±22.1 mV), strong electrostatic interactions between charged PLL and siRNA-loaded eEVs still led to successful PLL deposition and reversed the surface charge to a zeta potential of 33.3±1.6 mV. FIG. 10B shows that the hydrodynamic particle diameter slightly increased with each addition of charged polymer during the assembly process. Interestingly, the particle size of siRNA-encapsulated LbL-eEVs did not always increase with sequential deposition of polyelectrolytes. After the first layer PLL deposition, the addition of PAA decreased the overall size of vesicles. This may be due to strong ionic interactions between PAA and the PLL-siRNA-eEV complex, resulting in the formation of a dense polyelectrolyte coating. Another explanation for the varied size of PAA-eEV-siRNA is the alteration of the composition within the polymer-eEV complex following PAA assembly, as a result of the decreased diameter (87).

To characterize the cargo loading capacity and the influence of LbL assembly on cargo retention, siRNA loading within the eEVs was quantified after each step of the LbL process. Three commonly used electroporation buffers were also tested for siRNA loading, including Opti-MEM, 50 mM Trehalose and hypotonic electroporation buffer. FIG. 10C shows the percent of loaded siRNA remaining following each coating step. siRNA encapsulation into lipid-hybridized eEVs with a loading efficiency of 0.6 nmol siRNA within 10¹³ particles of zwitterionic POPC-doped eEVs was demonstrated previously. The quantity of siRNA loaded in the present work is comparable to prior work. Interestingly, while loading with hypotonic buffer improved siRNA retention during the earlier steps of LbL coating, only ˜30% of the loaded siRNA could be retained by the final step, regardless the buffer used. The decrease in the amount of loaded siRNA after LbL coating can potentially be attributed to the disassociation of affiliated siRNA surrounding eEVs, since a challenge of bulk electroporation systems is that a portion of siRNAs may not be entirely encapsulated into the vesicles post-electroporation and may not be fully removed after the wash-purification procedure (87). The effects of siRNA loading in various buffers on gene silencing efficacy are in Example 9.

The small molecule drug DOX was in the polyelectrolyte multilayer shell during the PAA coating step by forming PAA-DOX complexes via electrostatic interactions (37). To understand the effects of concentration on DOX loading into the polyelectrolyte shells, a concentration range of 0-1000 μg/ml was investigated. FIG. 10D shows that DOX loading increased in an exponential growth manner and reached saturation at 400 μg/ml. The maximum loading achieved after complexing the final layer of PBAE was 155±5 ng DOX per 10¹¹ vesicles. Based on these results, concentrations of 0.3 mg/ml and 0.6 mg/ml DOX (the saturated plateau) were selected as low and high dose groups, respectively, for the comparison of dose effects in subsequent studies.

### EXAMPLE 8

### Tumor Cell Selectivity of the LbL-eEVs

Intracellular delivery is essential for achieving the desired therapeutic effects, but selectivity of the delivery system for tumor cells is also critical to reduce cytotoxicity to normal cells. Therefore, the uptake efficiency of LbL-eEVs labeled with Deep Red dye in lung adenocarcinoma cells (A549) and non-cancerous lung fibroblasts (CCL-210) was investigated by confocal microscopy and flow cytometry quantification. However, prior to studying LbL-eEV uptake, a set of control liposomes to evaluate differences in uptake activity between the A549 and CCL-210 cells was used. Specifically, cationic DOTAP liposomes, zwitterionic POPC liposomes and anionic POPG liposomes were stained with Deep Red dye delivered to the cells. Interestingly, while the A549 cells exhibited approximately ˜2-fold higher uptake efficiency of zwitterionic liposomes, uptake of the DOTAP and POPG liposomes was greater in the CCL-210 cells. Thus, while the uptake mechanisms may differ, neither cell line exhibited consistently higher uptake activity.

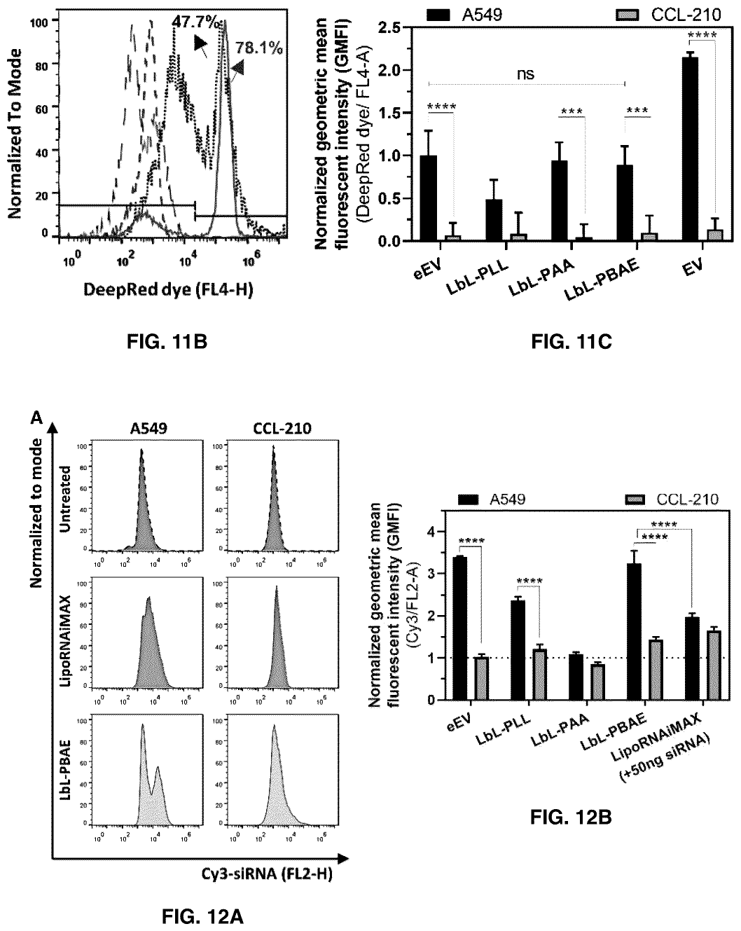

The confocal images in FIG. 11A demonstrate extensive uptake of the LbL-eEVs by the A549 cells. In contrast, minimal uptake was observed in the CCL-210 cells. The corresponding merged brightfield images showed the LbL-eEVs were mainly located in cytoplasm. FIG. 11B shows the flow cytometry histogram of FL4-H (Deep Red dye). A binary gating analysis was set to define LbL-eEV positive and negative cells, and it was found that LbL-eEVs were internalized by 78.1% of A549 cells compared to only 47.7% of CCL-210 cells. However, this gating analysis is inherently binary. To provide greater insight on LbL-eEV uptake, FIG. 11C shows the quantitative flow cytometry results of normalized geometric mean of fluorescence intensity (nGMFI) for LbL-eEVs with different polymer coatings (i.e., only PLL, PLL/PAA, and PLL/PAA/PBAE). While the prior work on native EVs demonstrated 15.8 times greater uptake of EVs in A549 cells compared to CCL-210 cells, the data obtained here show that the internalization of eEVs in A549 cells remains 14.2-fold greater than that in CCL-210 cells. Thus, the eEVs exhibited inherent selectivity for the cancer cells. Surprisingly, uptake of the LbL-PBAE-eEVs in the A549 cells was not reduced when compared to eEVs. Moreover, a LbL-eEV uptake was 5.2-fold higher in A549 cells compared to CCL-210 cells, indicating that the selectivity of the eEVs was not compromised by the LbL coating. This result can likely be attributed to the exposure of tetraspanin proteins or exosomal integrins, which have been reported to direct the selective internalization of EVs in different cells (89).

### EXAMPLE 9

### Potential Antitumor Efficacy of LbL-eEVs

### SiRNA Delivery and Gene Silencing

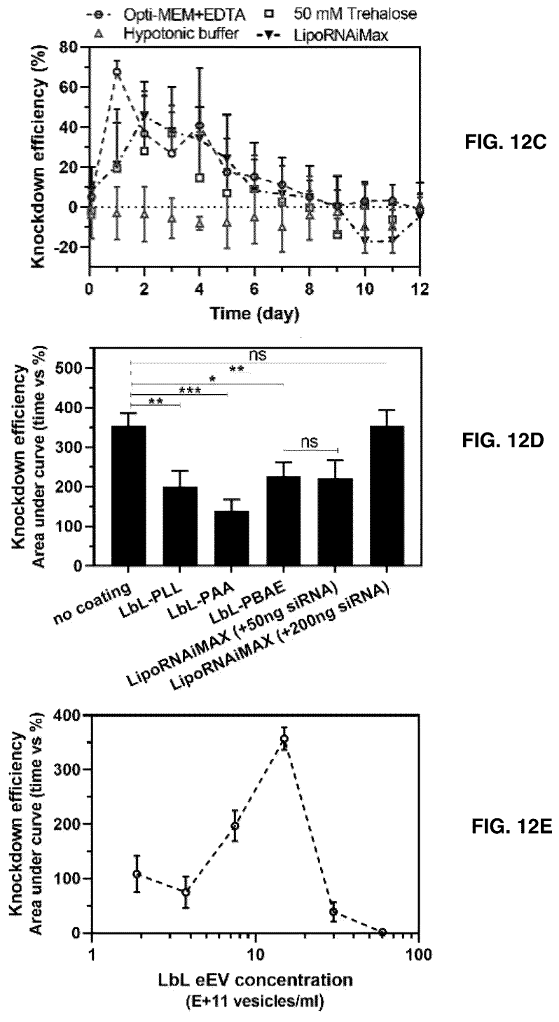

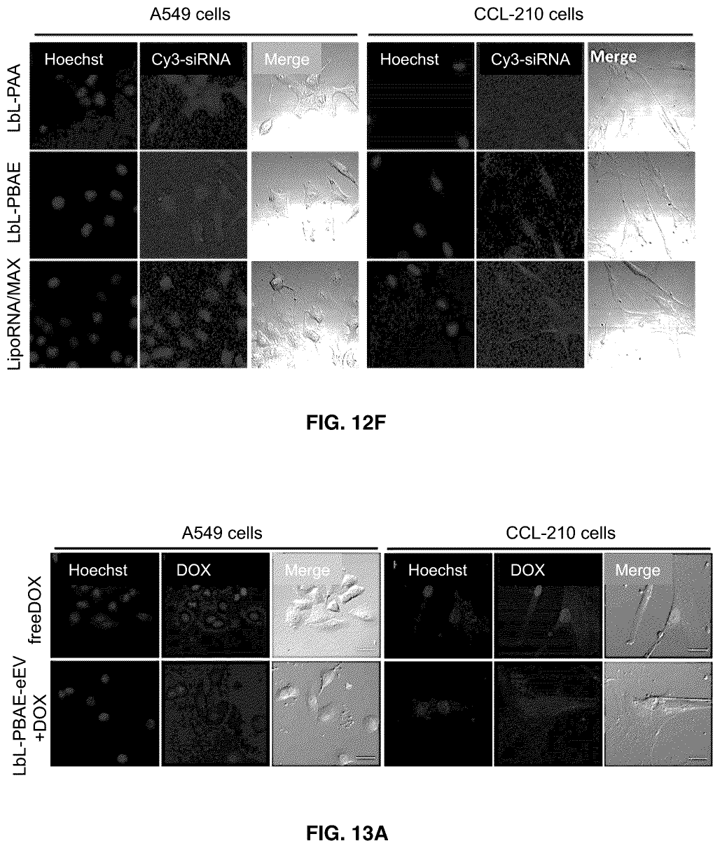

Efficient cellular uptake and endosomal escape are crucial to translocate siRNA to the cytoplasmic region for RNAi-triggered gene silencing. To demonstrate the cargo siRNA within LbL-eEVs can be delivered intracellularly, Cy3-labelled siRNA was loaded in LbL-eEVs to evaluate the internalization of siRNA in both A549 and CCL-210 cells. Commercial RNAi silencing reagent LipofectamineRNAiMAX was used as a positive control and the uptake efficiency of siRNA was subsequently quantified by flow cytometry and comparisons of nGMFI (FIGS. 12A-12F). The confocal microscopy images of Cy3-siRNA internalization are shown in FIG. 12F.

The histogram of FL2-H (Cy3-siRNA) (FIG. 12A) confirms significantly enhanced uptake of LbL-PBAE-eEVs in A549 cells. Notably, the nGMFI in FIG. 12B shows LbL-PBAE-eEVs more efficiently delivered siRNA intracellularly compared to other polyelectrolyte layered-eEVs. Moreover, the uptake amounts of Cy3-siRNA delivered by LbL-PBAE-eEVs in A549 was 2.27-fold higher than that in CCL-210 cells, whereas no significant difference between the uptake efficiency in A549 and CCL-210 cells was observed for the commercial lipofectamine RNAiMAX transfection reagents. These findings further confirm the potential of LbL-PBAE-eEVs for preferential delivery to tumor cells.

To validate that delivered siRNA can effectively suppress gene expression, siRNA against green fluorescence protein (siRNA-GFP) was delivered to GFP-expressing A549 cells. This model gene approach allowed for ease of monitoring as well as quantitative analysis of efficacy based on GFP fluorescence intensity. FIG. 12C shows the knockdown efficiency in GFP expression over time in the cells treated with LbL-eEVs loaded using 3 different electroporation buffers. LbL-eEVs in Opti-MEM resulted in the most effective silencing, and the 67.5±5.5% knockdown efficiency on day 2 after delivery was a 3.5-fold higher than the other formulations. On day 4, the knockdown efficiency of the LbL-eEV loaded using Opti-MEM was 40.9±24.8%, which was comparable to the knockdown efficiency of commercial Lipofectamine RNAiMax with 50 ng siRNA delivery (34.0±16.2%).

To investigate the influence of different layered LbL-eEVs on knockdown efficiency, the same electroporation procedure was repeated in Opti-MEM media. The decrease of GFP expression in the cells was quantified by integrating the area under the curve (knockdown efficiency over time), as shown in FIG. 12D. The overall knockdown efficiency results show that the gene silencing capability of eEVs without polyelectrolyte shells was significantly higher than that of LbL-eEVs. However, the PBAE-layered eEVs were still able to effectively silence gene expression to the same degree compared with Lipofectamine treatment (50 ng siRNA/100 μl). Importantly, only about 0.6 nmol siRNA (˜10 ng) was encapsulated in 10¹³ eEVs. Thus, matching the efficacy of Lipofectamine indicates that the instant LbL-eEVs are still a highly effective gene delivery platform.

The knockdown efficiency of LbL-eEVs at varying doses was evaluated, as shown in FIG. 12E. The results demonstrate that gene silencing increased with increasing vesicle/siRNA concentration and reached a maximum knockdown efficiency at a concentration of 1.5×10¹² vesicles/ml. The knockdown efficiency decreased drastically at higher siRNA dose. This is likely due to the off-target effects of siRNA at higher dose, leads to the reduction of GFP expression in the control group treated with scramble siRNA, and therefore, limits the dose of siRNA can be applied. Consequently, the concentration of 1.5×10¹² vesicles/ml of LbL-eEV/siRNA complexes, exhibiting the maximum efficacy, was further used in the following co-delivery experiments.

### DOX Delivery and Cancer Killing Efficiency

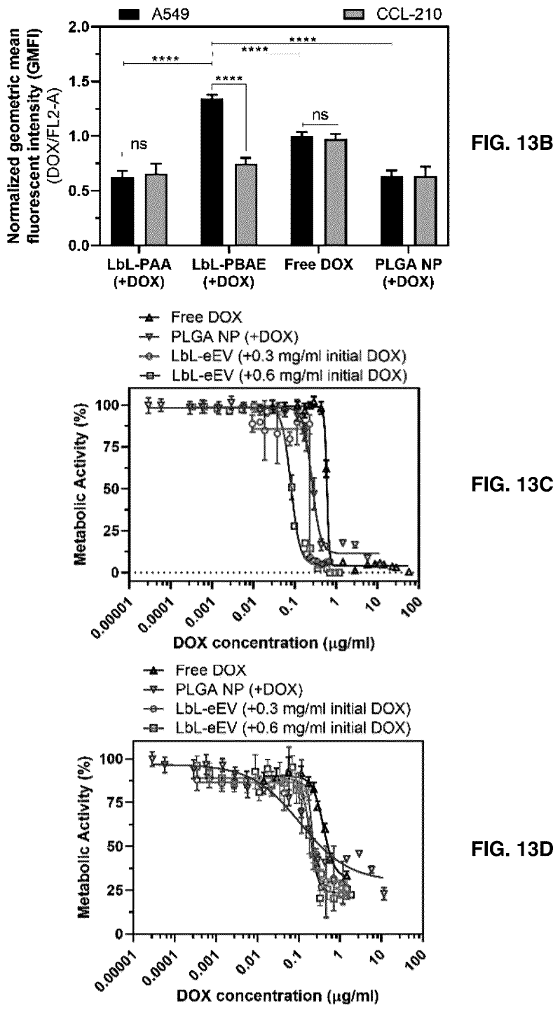

The primary mechanism of anti-tumor activity from DOX is the intercalation of DOX molecule into DNA, leading to inhibition of DNA synthesis. Therefore, it is necessary for DOX molecules to enter cell nuclei for therapeutic efficacy. Cellular internalization of DOX was evaluated by the same procedure as in the prior sections. FIG. 13A demonstrates free DOX enters cell nuclei as a DNA intercalation agent for the induction of programmed cell death. Notably, the delivery of DOX by LbL-eEVs to CCL-210 showed decreased nuclear localization compared to in A549 cells. To further quantitively assess the preferential uptake and DOX delivery with LbL-eEVs, DOX-loaded poly (lactic-co-glycolic acid) nanoparticles (PLGA NPs), one of the most well-studied drug delivery systems, were tested using the same procedure for comparison. Consistent with the prior siRNA uptake results, DOX delivery by LbL-PBAE-eEVs resulted in significantly higher internalization in A549 cells (1.79-fold) than in CCL-210 cells, as shown in FIG. 13B. In contrast, synthetic PLGA nanoparticles showed no significant difference in DOX delivery to A549 and CCL-210 cells, confirming the potential for preferential delivery to tumor cells with the LbL-eEV system. It is necessary to point out that the uptake efficiency (DOX) was significantly improved by LbL-PBAE, which was 2.15-fold higher than that by LbL-PAA, suggesting the necessity of the final cationic layer to facilitate cellular internalization. These results were also consistent with the siRNA internalization results in the previous section.

Next a cell proliferation assay (MTS assay) was used to evaluate the efficacy of DOX delivery with different formulations in cells. Prior to the experiment, the dose-dependent cytotoxicity of blank LbL-eEVs was examined in both A549 and CCL-210 cells. No toxicity was observed in the concentration range of 0-22×10¹² particles/ml after 3 days incubation, confirming cytocompatibility of the vesicles. Subsequently, the same experiment was repeated for DOX-loaded LbL-eEVs. FIGS. 13C-13D show the dose-responsive curves of free DOX, DOX-loaded PLGA NPs, and DOX-loaded LbL-eEVs in A549 and CCL-210 cells, respectively. The LbL-eEVs were loaded using a low or high concentration of DOX (0.3 and 0.6 mg/ml, respectively), based on the loading results (FIG. 10D). For all treatment groups, the DOX amount in LbL-eEVs administered to the cells was quantified spectrophotometrically. The quantified inhibitory concentration (IC) values of DOX delivered by the various carriers are presented in Table 2. Both PLGA NPs and LbL-eEVs with DOX showed a higher antitumor efficacy at lower IC compared to free DOX, which can be attributed to the enhanced cellular internalization of drugs. Among all the therapeutic groups, LbL-eEVs prepared with the 0.6 mg/ml loading concentration of DOX were most effective, indicating the cancer killing efficiency depends on the amount of DOX complexed in the polyelectrolyte layers. In contrast to the conventional PLGA system, the IC₅₀ of LbL-eEV (+0.6 mg/ml DOX) was 3.2-fold decreased in A549 cells, indicating that LbL-eEV delivery system requires a substantially lower dose of DOX to achieve the same cancer cell killing efficiency. While the IC values in CCL-210 were also reduced, the IC values for LbL-eEV mediated DOX delivery were at least 1.8-fold lower in A549 cells than in CCL-210 cells.

TABLE 2
Inhibitory concentration (IC) values of DOX

| | | Inhibitory concentration (μ/ml) | | |
|---|---|---|---|---|
| Cell line | Treatment | IC₁₀ | IC₅₀ | IC₉₀ |
| A549 | Free DOX | 0.510 | 0.598 ± 0.008 | 0.690 |
| | PLGA NP | 0.152 | 0.259 ± 0.007 | 0.440 |
| | LbL-eEV + 0.3 mg/ml | 0.230 | ~0.232 | 0.233 |
| | initial DOX | | | |
| | LbL-eEV + 0.6 mg/ml | 0.050 | 0.081 ± 0.002 | 0.140 |
| | initial DOX | | | |
| CCL210 | Free DOX | 0.200 | 0.400 ± 0.024 | 0.780 |
| | PLGA NP | 0.005 | 0.109 ± 0.021 | 2.210 |
| | LbL-eEV + 0.3 mg/ml | 0.162 | 0.213 ± 0.008 | 0.279 |
| | initial DOX | | | |
| | LbL-eEV + 0.6 mg/ml | 0.115 | 0.189 ± 0.006 | 0.310 |
| | initial DOX | | | |

In Table 2 IC10, IC50 and IC90 values are calculated from inhibitor curves for free DOX, PLGA nanoparticles (PLGA NP), and LbL-eEVs loaded using low (0.3 mg/ml) and high (0.6 mg/ml) concentrations of DOX in A549 and CCL-210 cells. All the sigmoidal concentration response curves were fitted with Hill's equation using GraphPad software. Due to the high steep curve (large absolute value of Hill's slope), an ambiguous estimate (wide confidence interval) of the IC50 value (˜0.232) was obtained in the fitting results of LbL-eEV+0.6 mg/ml initial DOX treated in A549 cells.

### EXAMPLE 10

### Co-Delivery of siRNA and DOX

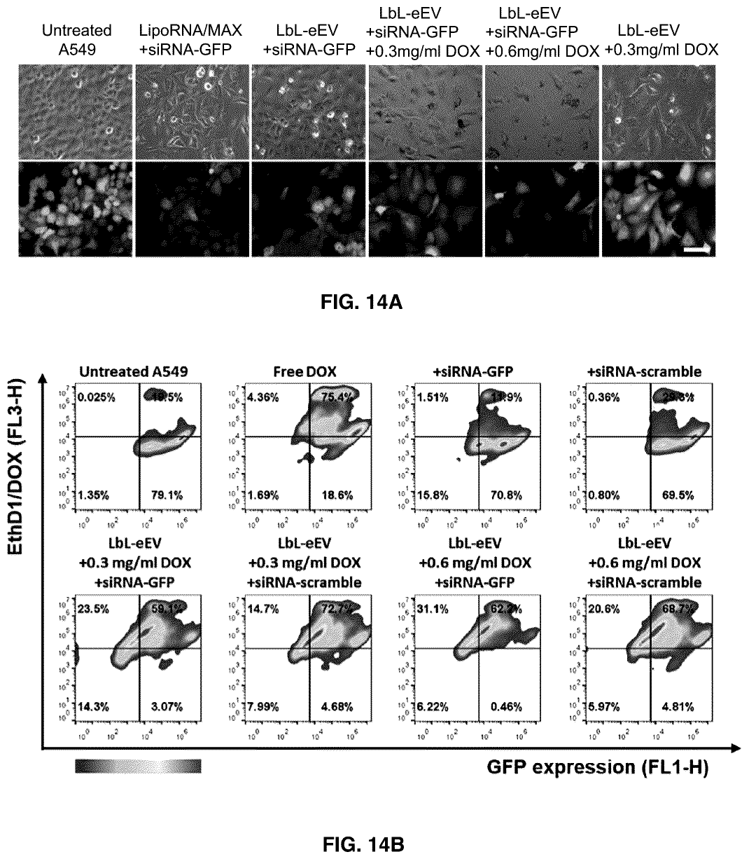

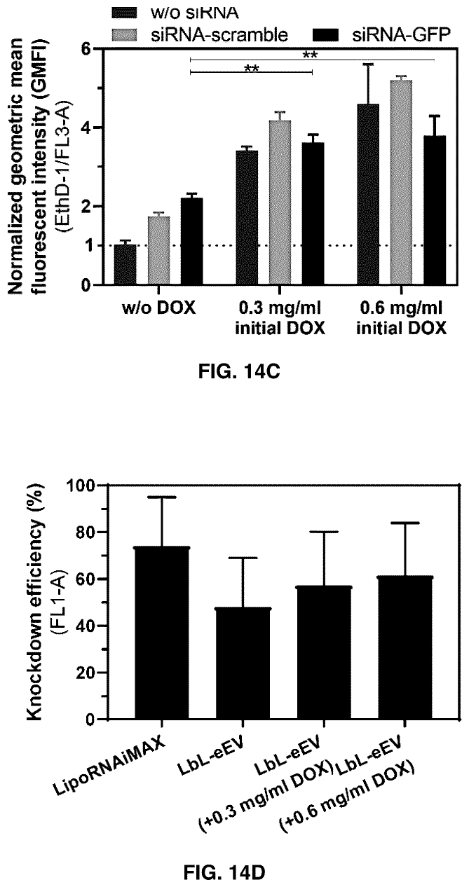

The experimental results above demonstrated that LbL-eEVs are effective for preferential delivery of siRNA and DOX to A549 cancer cells separately. To confirm the utility of LbL-eEVs as a co-delivery system, GFP-expressing A549 cells were treated with LbL-eEVs and then quantitatively analyzed by flow cytometry. The cells were also stained with ethidium homodimer-1 (EthD-1) to assess the effects of DOX delivery. LbL-eEVs containing siRNA alone, DOX alone, and both siRNA and DOX. LbL-eEVs prepared with two different DOX loading concentrations were also tested (note: the overall DOX dose was held constant). Treatments with siRNA-GFP and control scramble siRNA were prepared in pairs for each individual condition. FIG. 14A shows the qualitative results of A549 cells with LbL-eEV mediated co-delivery of SiRNA/DOX. Morphological changes were not observed in the cells with the delivery of either siRNA-GFP or control scramble siRNA, but considerable cell enlargement was observed in the DOX-delivery groups. On the other hand, remarkably reduced green fluorescence was observed in the groups treated with siRNA-GFP (i.e., Lipofectamine+siRNA-GFP, LbL-eEV+siRNA-GFP), indicating downregulation of GFP expression. The cell images in co-delivered formulations demonstrate both morphological changes and reduced green fluorescence in the cells, which verifies the co-delivery of siRNA and DOX. FIG. 14B shows quadratic graphs from the flow cytometry analysis. The upper-right quadrant (Q1) represents GFP-positive and EthD-1/DOX positive cells. The upper left quadrant (Q2) corresponds to cells that are positive only for EthD-1/DOX. The bottom-left quadrant (Q3) represents double-negative cells. The bottom-right quadrant (Q4) represents cells that are positive only for GFP. The results show that siRNA-GFP/DOX co-delivery by LbL-eEVs markedly increased the percentages of cells located in Q1 and Q2 compared to untreated cells. The percentage of cells in Q2 was 23.5% and 31.1% in the siRNA-GFP/0.3 mg/ml DOX and siRNA-GFP/0.6 mg/ml DOX groups, respectively. In contrast, only 14.7% and 20.6% of the cells were in Q2 for the siRNA-scramble/0.3 mg/ml DOX and siRNA-scramble/0.6 mg/ml DOX groups, respectively. While these results provide evidence that the LbL-eEV particles achieved synchronous delivery of siRNA and DOX, further analysis was performed based on normalized GMFI measurements. Analysis of the FL3 channel indicated that DOX delivery was not hampered by the co-delivery of SIRNA (FIG. 14C). Moreover, the knockdown efficiency of single cells treated with SIRNA/DOX co-loaded formulations was found to be comparable to the siRNA alone group (FIG. 14D). Collectively, the results of these experiments confirm that co-loading siRNA and DOX does not compromise the efficacy of the LbL-eEV delivery system and that effective target gene silencing and DOX delivery are achieved simultaneously.

The following references are cited herein.
1. Gilligan, K. E. and Dwyer, R. M., International Journal of Molecular Sciences 18:1122, 2017.
2. Harding et al. Journal of Cell Biology, 200:367-371, 2013.
3. Jiang, X.-C. and Gao, J.-Q. J, International Journal of Pharmaceutics, 521:167-175, 2017b.
4. Andaloussi et al. Nature Reviews Drug discovery 12:347-357, 2013.
5. Srivastava et al. 12:1159-1173, 2016.
6. Armstrong et al. ACS Nano 11, 69-83, 2017.
7. Luan et al. Acta Pharmacologica Sinica 38:754, 2017.
8. Jiang, X.-C. and Gao, J.-Q., International Journal of Pharmaceutics 521:167-175, 2017.
9. Li et al. ACS Biomaterials Science & Engineering, 5(10):4870-4881, 2019.
10. Alhasan et al. Small 10(1):186-192, 2014.
11. Kim et al. Nanomedicine: Nanotechnology, Biology and Medicine, 12(3):655-664, 2016.
12. Andaloussi et al. Advanced drug delivery reviews 65:391-397, 2013.
13. Maheshwari et al. Nanomedicine 12:2653-2675, 2017.
14. Tekade et al. Drug Discovery Today 21(11):1761-1774, 2016.
15. Wang et al. Colloids and Surfaces B: Biointerfaces, 157:297-308, 2017.
16. Yoon et al. Scientific Reports, 4:6878, 2014.
17. Jiang et al. European Journal of Pharmaceutics and Biopharmaceutics 2010, 76(2):170-178, 2010.
18. Sakurai et al. Molecular Pharmaceutics, 11(8):2713-2719, 2014.
19. Tsouris et al. Biotechnology Advances, 32(5):1037-1050, 2014.
20. Zhang et al. Journal of Controlled Release, 240:489-503, 2016.
21. Bhise et al. Small, 8(3):367-373, 2012.
22. Taratula et al. Journal of Controlled Release, 171(3):349-357, 2013.
23. Chen et al. RSC Advances, 8(32): 17710-17722, 2018.
24. Xia et al. International Journal of Nanomedicine, 13:143-159, 2018.
25. Cheng et al. ACS Applied Materials & Interfaces, 8(7):4347-4356, 2016.
26. Dong et al. Biomaterials 2013, 34(20), 4849-4859, 2013.
27. Gao et al. ACS Applied Materials & Interfaces, 11(18):16296-16310, 2019.
28. Jia et al. Journal of Controlled Release, 216:9-17, 2015.
29. Yin et al. Biomaterials, 61:10-25, 2015.
30. Armstrong, J. P. and Stevens, M. M. Advanced Drug Delivery Review, 130:12-16, 2018.
31. Fuhrmann et al. J.A.M., 30:1706616, 2018.
32. Greening et al. Proteomic Profiling. Springer, pp. 179-209, 2015.
33. Szatanek et al. International Journal of Molecular Medicine, 36:11-17, 2015.
34. Bishop et al. Acta Biomaterialia, 11:393-403, 2015.
35. Am Hong et al. Scientific Reports, 8(1): 1-7, 2018.
36. Wohl, B. M. and Engbersen, J. F., Journal of Controlled Release, 158(1):2-14, 2012.
37. Kitaeva et al. Langmuir, 20(16):6575-6579, 2004.
38. Chen et al. Journal of Nanomaterials, doi.org/10.1155/2013/345738, 2013.
39. Bishop et al. Journal of Biomedical Materials Research Part A, 104(3):707-713.40, 2016.
40. Routkevitch et al. ACS Biomaterials Science & Engineering, 6(6):3411-3421, 2020.
41. Kozielski et al. ACS Nano, 8(4):3232-3241, 2014.
42. Bishop et al. Acta Biomaterialia, 11:393-403, 2015.
43. Löf et al. Scientific Reports, 6:34358, 2016.
44. Properzi et al. Biomarkers in Medicine 7:769-778, 2013.
45. Théry et al. Current Protocols in Cell Biology, 3.22:21-23, 29, 2006.
46. Osteikoetxea et al. PLOS One, 10:e0121184, 2015.
47. El-Andaloussi et al. Nature Protocols, 7:2112, 2012.
48. Alvarez-Erviti et al. Nature Biotechnology, 29:341-345, 2011.
49. Golzio et al. Biophysical Journal, 74:3015-3022, 1998.
50. Usaj, M. and Kanduser, M. J. Journal of Membrane Biology, 245:583-590, 2012.
51. Ušaj et al. Oncology, 43:108-119, 2009.
52. Lamichhane et al. Molecular Pharmaceutics, 12:3650-3657, 2015.
53. Navakanitworakul et al. Scientific Reports 6:25486, 2016.
54. Haraszti et al. Molecular Therapy, 26(12):2838-2847, 2018.
55. Kamerkar et al. Nature, 546(7659):498-503, 2017.
56. Pi et al. J.N.n., 13:82, 2018.
57. Gregory, J. J. Adv Colloid Interface Sci, 147:09-123, 2009.
58. Jamaluddin, M. P. and Krishman, L. K. J. Biochem. Bioph. Methods, 14(4):191-200, 1987.
59. Lu et al. International Journal of Pharmaceutics, 550:100-113, 2018.
60. Zhang et al. J.J.o.C.R., 197:208-218, 2015.
61. Hood et al. Analytical Biochemistry 448:41-49, 2014.
62. Tran et al. Clinical Immunology 160:46-58, 2015.
63. Jhan et al. International Journal of Pharmaceutics, 573:118802, 2020.
64. Takov et al. Journal of Extracellular Vesicles, 6(1):1388731, 2017.
65. Betancourt et al. Nanomedicine (London, England), 2(2):219-32, 2007.
66. Jang et al. ACS Nano, 7:7698-7710, 2013.
67. Christianson et al. PNAS, 201304266, 2013.
68. Rana et al. 2012. Int. J. Biochem. Cell Biol., 44:1574-1584, 2012.
69. Yeo et al. Advanced Drug Delivery Review, 65:336-341, 2013.
70. Turinetto et al. Int J Mol Sci, 17:1164, 2016.
71. Jin et al. Int J Mol Sci, 14:7986-18001, 2013.
72. Kanada et al. Proceedings of the National Academy of Sciences, 201418401, 2015.
73. Sun et al. Advanced Drug Delivery Reviews 65:342-347, 2013.
74. Mitchell et al. Journal of Immunological Methods, 335:98-105, 2008.
75. Watson et al. J.B., 105:195-205, 2016.
76. Antimisiaris et al. International Journal of Pharmaceutics 525, 293-312, 2017.
77. Piffoux et al. ACS Nano, 12:6830-6842, 2018.
78. Sato et al. Scientific Reports, 6:21933, 2016.
79. Ren et al. Biomaterials Science, 4:910-921, 2016.
80. Marimpietri et al. PloS one, 8:e75054, 2013.
81. Scherer et al. The AAPS Journal, 2012. 14:236-243, 2012.
82. Stapulionis, R. J. B., Bioenergetics, 48:249-254, 1999.
83. Kooijmans et al. Journal of Controlled Release, 172(1):229-238, 2013.
84. Cho et al. Journal of Medicinal Chemistry, 6:385, 2011.
85. Sui et al. Langmuir, 19(6):2491-2495, 2003.
86. Seyrek et al. Physical Chemistry Chemical Physics, 13(28):12716-12719, 2011.
87. Ramasamy et al. Carbohydrate Polymers, 102:653-661, 2014.
88. Vader et al. Advanced Drug Delivery Reviews, 106:148-156, 2016.
89. Qiao et al. Theranostics, 10(8):3474-3487, 2020.

What is claimed is:

1. An extracellular vesicle delivery vehicle, comprising:
at least one lipid hybridized with a membrane of the extracellular vesicle;
a nucleic acid loaded within a core of the extracellular vesicle;
a multi-layered polyelectrolyte coating deposited around the extracellular vesicle; and
a therapeutic drug complexed with the multi-layered polyelectrolyte coating.

2. The engineered extracellular vesicle of claim 1, wherein the lipid is a synthetic lipid comprising 1-Palmitoyl-2-oleoyl-sn-glycero-3-phosphocholine (POPC), 1-Palmitoyl-2-oleoyl-sn-glycero-3-phosphoglycerol (POPG), dipalmitoylphosphatidylcholine (DPPC), or, 1,2-dipalmitoyl-sn-glycero-3-phosphocholine (DOTAP) or a combination thereof.

3. The engineered extracellular vesicle of claim 1, wherein the nucleic acid is a synthetic DNA, a naturally occurring DNA, a synthetic RNA, or a naturally occurring RNA, or fragments thereof.

4. The engineered extracellular vesicle of claim 3, wherein the synthetic RNA or naturally occuring RNA is a small-interfering RNA (siRNA) or a microRNA (miRNA).

5. The extracellular vesicle delivery vehicle of claim 1, wherein the multi-layered polyelectrolyte coating comprises alternating layers of oppositely charged polyelectrolytes.

6. The extracellular vesicle delivery vehicle of claim 5, wherein the oppositely charged polyelectrolytes are poly-L-lysine, polyacrylic acid or poly-β-amino ester or other anionic polyelectrolyte or cationic polyelectrolyte structured for oppositely charged complexation.

7. The extracellular vesicle delivery vehicle of claim 1, wherein the therapeutic drug is an anti-cancer drug.

8. The extracellular vesicle delivery vehicle of claim 7, wherein the anti-cancer drug is an aptamer, an antibody, a duobody or other therapeutic protein, or a small molecule drug.

9. A method for preparing an extracellular vesicle delivery vehicle, comprising the steps of:
culturing biological cells in vitro in a culture medium;
isolating the extracellular vesicles from the biological cells;
extruding the isolated extracellular vesicles with at least one lipid to form a lipid-hybridized extracellular vesicle;
loading a nucleic acid into a core of the lipid-hybridized extracellular vesicle;
depositing, sequentially, a first layer of a polycation to coat the lipid-hybridized extracellular vesicle, a second layer of a polyanion onto the first layer and a third layer of a cationic polymer ester onto the second layer; and
complexing a therapeutic drug to the second layer to form the extracellular vesicle delivery vehicle.

10. The method of claim 9, wherein the biological cell is a cancer cell.

11. The method of claim 9, wherein the lipid is a synthetic lipid comprising 1-palmitoyl-2-oleoyl-sn-glycero-3-phosphocholine (POPC), 1-palmitoyl-2-oleoyl-sn-glycero-3-phosphoglycerol (POPG), dipalmitoylphosphatidylcholine (DPPC), or, 1,2-dipalmitoyl-sn-glycero-3-phosphocholine (DOTAP) or a combination thereof.

12. The method of claim 9, wherein the polycation is poly(L-lysine), the polyanion is poly(acrylic acid) and the cationic polymer ester is poly(β-amino ester).

13. The method of claim 9, wherein the nucleic acid is a synthetic DNA, a naturally occurring DNA, a synthetic RNA, or a naturally occurring RNA, or fragments thereof.

14. The method of claim 9, wherein the therapeutic drug is an anti-cancer drug comprising an aptamer, an antibody, a duobody or other therapeutic protein, or a small molecule drug.
